(19) United States
(12) Patent Application Publication
(10) Pub. No.: US 2024/0141003 A1
(43) Pub. Date: May 2, 2024

(54) MICROBIAL PRODUCTION OF POLYMERIC AMYLOID FIBERS

(71) Applicant: Washington University, St. Louis, MO (US)

(72) Inventors: Fuzhong Zhang, St. Louis, MO (US); Jingyao Li, St. Louis, MO (US)

(21) Appl. No.: 18/278,707

(22) PCT Filed: Feb. 25, 2022
(86) PCT No.: PCT/US2022/017828

§ 371 Date: Aug. 24, 2023

Related U.S. Application Data
(60) Provisional application No. 63/153,792, filed on Feb. 25, 2021.

(51) Int. Cl. C07K 14/47; C07K 14/435; D01F 4/00

(52) U.S. Cl. CPC C07K 14/4711; C07K 14/43518; D01F 4/00; C07K 2319/00; D10B 2401/063

(57) ABSTRACT

The present disclosure is directed to systems and methods for synthesizing a recombinant polymeric amyloid and recombinant polymeric amyloid fibers. In some embodiments, the methods comprise synthesizing tandem repeats of an amyloid peptide and a glycine-rich linker peptide in vivo in a heterologous host. In other embodiments, the recombinant polymeric amyloid fibers comprise a plurality of polymeric amyloid fibrils each comprising a plurality of β-sheet crystals, wherein the β-sheet crystals comprise tandem repeats of an amyloid peptide and a glycine-rich linker peptide, and wherein the plurality of β-sheet crystals are aligned in parallel with a fiber axis.

### STATEMENT REGARDING FEDERALLY SPONSORED RESEARCH & DEVELOPMENT

This invention was made with government support under 20196702129943 awarded by the United States Department of Agriculture (USDA) and N000141912126 awarded by the Office of Naval Research (ONR). The government has certain rights in the invention.

### CROSS-REFERENCE TO RELATED APPLICATIONS

This application claims the benefit of priority to U.S. Prov. Pat. App. No. 63/153,792 filed Feb. 25, 2021, which is incorporated by reference herein in its entirety.

### SEQUENCE LISTING

The instant application contains a sequence listing in paper format and in computer readable format, the teachings and content of which are hereby incorporated by reference.

### FIELD OF THE DISCLOSURE

The field of the disclosure relates generally to microbially-produced fibers. More specifically, the field of the disclosure relates to microbial production of polymeric amyloid fibers having gigapascal tensile strength.

### BACKGROUND OF THE DISCLOSURE

Amyloids represent a large group of structural proteins that form highly ordered cross-β protofilaments in which β-strands align and are perpendicular to the fibril axis. Networks of non-covalent interactions between neighboring β-strands through hydrogen bonding and between adjacent β-sheets through electrostatic interaction, π-π stacking, and hydrophobic effects, confer useful mechanical properties to these amyloid nanofibrils.

Although some amyloids are known for their roles associated with neurodegenerative diseases, the discovery of nonpathogenic but functional amyloids, such as curli fibrils in Escherichia coli, silkmoth chorion proteins in silkmoth eggshell, and catalytic scaffold Pmel17 in humans, has drawn increasing attention to their potential in material applications. For example, considerable efforts have been made to use amyloid peptides in bioadhesives, biomineralizations, biosensors, and carriers for controlled drug deliveries. However, few attempts have been made to translate the mechanical properties at nanoscale into macroscopic materials with equivalent properties, because amyloid proteins often fail to form an extensive and strong interactive network at macroscales.

Dragline spider silk is one of the strongest and toughest natural macroscopic materials. This unique combination of high strength and toughness makes the production of recombinant silk fibers highly desirable. However, achieving gigapascal tensile strength with higher than 150 MJ/m⁻³ toughness has proven to be extremely difficult. As a semi-crystalline material, dragline silk fibers contain β-sheet nano-crystallites formed by poly-alanine sequences and amorphous domains arising from flexible peptide sequences, such as flexible glycine-rich sequences. The crystallinity of the material positively affects fiber tensile strength. While natural spider silk has 28-44% crystallinity, recombinant silk fibers generated from artificial spinning processes have significantly lower crystallinity. The lower crystallinity in recombinant fibers can be attributed to multiple factors, such as different spinning conditions used in artificial spinning, which is extremely difficult to overcome due to the sophisticated natural spinning process employed by spiders.

Accordingly, there is a need for recombinant, macroscopic fiber materials that exhibit comparable mechanical performance to spider silk fibers. The embodiments described herein resolve at least these known deficiencies.

### BRIEF DESCRIPTION OF THE DISCLOSURE

In one aspect, the present disclosure is directed to a system for synthesizing a recombinant polymeric amyloid in vivo. The system comprises a host cell and a plasmid encoding tandem repeats of an amyloid peptide and a glycine-rich linker peptide.

In some embodiments, the glycine-rich linker peptide is a silk amino acid sequence or other flexible peptide sequence. In some embodiments, the amyloid peptide is selected from a full amyloid peptide and an amyloid peptide fragment. In some embodiments, the amyloid peptide is selected from a parallel amyloid, an antiparallel homo-facial amyloid, and an antiparallel hetero-facial amyloid. In some embodiments, the host cell is a microbial cell. In some embodiments, the plasmid encodes at least about 16 tandem repeats. In some embodiments, the amyloid peptide is encoded by an amino acid sequence having at least about 50% similarity with a β-sheet-forming amyloid peptide. In some embodiments, the amyloid peptide is encoded by an amino acid sequence having an identical sequence similarity with a β-sheet-forming amyloid peptide.

In another aspect, the present disclosure is directed to a method for synthesizing a recombinant polymeric amyloid. The method comprises synthesizing tandem repeats of an amyloid peptide and a glycine-rich linker peptide in vivo in a heterologous host.

In some embodiments, the amyloid peptide is selected from a parallel amyloid, an antiparallel homo-facial amyloid, and an antiparallel hetero-facial amyloid. In some embodiments, the glycine-rich peptide is a silk amino acid sequence or other flexible peptide sequence. In some embodiments, the method further comprises purifying the recombinant polymeric amyloid. In some embodiments, the method further comprises spinning the recombinant polymeric amyloid into fibers. In some embodiments, the recombinant polymeric amyloid has a molecular weight of at least about 45 kDa.

In yet another aspect, the present disclosure is directed to a recombinant polymeric amyloid fiber. The fiber comprises a plurality of polymeric amyloid fibrils each comprising a plurality of β-sheet crystals, wherein the β-sheet crystals comprise tandem repeats of an amyloid peptide and a glycine-rich linker peptide, and wherein the plurality of β-sheet crystals are aligned in parallel with a fiber axis.

In some embodiments, the amyloid peptide is selected from a parallel amyloid, an antiparallel homo-facial amyloid, and an antiparallel hetero-facial amyloid. In some embodiments, the glycine-rich linker peptide is a silk amino acid sequence or other flexible peptide sequence. In some embodiments, the fiber has a crystallinity of at least about 10%. In some embodiments, the fiber has an ultimate tensile strength of at least about 0.90 GPa. In some embodiments, the β-sheet crystals comprise at least about 16 tandem repeats, at least about 48 tandem repeats, at least about 90 tandem repeats, or at least about 120 tandem repeats.

### BRIEF DESCRIPTION OF THE DRAWINGS

The embodiments described herein may be better understood by referring to the following description in conjunction with the accompanying drawings.

FIG. 1A is an exemplary embodiment of polymeric amyloid fibers used to form strong macroscopic fibers including design of polymeric amyloid fibers in accordance with the present disclosure. The β-sheet forming amyloid peptides are connected with flexible glycine-rich linkers from spidroins, building up a polymeric protein that contains tens or even hundreds of such tandem repeats. During the wet-spinning process, these amyloid peptides fold into β-sheet crystals well-aligned with fibril axis, conferring mechanical strength at the macroscale.

FIG. 1B is an exemplary embodiment of polymeric amyloid fibers used to form strong macroscopic fibers including crystal structures of self-assembled amyloid peptide in their cross-β forms in accordance with the present disclosure. GDVIEV, SEQ ID NO: 1 (PDB: 3SGS); KLVFFAE, SEQ ID NO: 2 (PDB: 3OW9); and FGAILSS, SEQ ID NO: 3 (PDB: 5E61).

FIG. 1C is an exemplary embodiment of polymeric amyloid fibers used to form strong macroscopic fibers including a representative stress-strain curve from tensile testing of 16× GDVIEV amyloid fibers in accordance with the present disclosure.

FIG. 1D is an exemplary embodiment of polymeric amyloid fibers used to form strong macroscopic fibers including a representative stress-strain curve from tensile testing of 16× KLVFFAE amyloid fibers in accordance with the present disclosure.

FIG. 1E is an exemplary embodiment of polymeric amyloid fibers used to form strong macroscopic fibers including a representative stress-strain curve from tensile testing of 16× FGAILSS amyloid fibers in accordance with the present disclosure.

FIG. 1F is an exemplary embodiment of polymeric amyloid fibers used to form strong macroscopic fibers including a representative stress-strain curve from tensile testing of 16× recombinant silk fibers in accordance with the present disclosure.

FIG. 1G is an exemplary embodiment of polymeric amyloid fibers used to form strong macroscopic fibers including ultimate tensile stress of 16× amyloid fibers and recombinant silk fibers in accordance with the present disclosure. Error bars represent standard deviation. *p<0.05, two-tailed unpaired t-test. For the 16× amyloid fibers, n=10, and for the recombinant silk fibers, n=6.

FIG. 1H is an exemplary embodiment of polymeric amyloid fibers used to form strong macroscopic fibers including Young's modulus of 16× amyloid fibers and recombinant silk fibers in accordance with the present disclosure. Error bars represent standard deviation. *p<0.05, two-tailed unpaired t-test. For the 16× amyloid fibers, n=10, and for the recombinant silk fibers, n=6.

FIG. 1I is an exemplary embodiment of polymeric amyloid fibers used to form strong macroscopic fibers including breaking strain of 16× amyloid fibers and recombinant silk fibers in accordance with the present disclosure. Error bars represent standard deviation. *p<0.05, two-tailed unpaired t-test. For the 16× amyloid fibers, n=10, and for the recombinant silk fibers, n=6.

FIG. 1J is an exemplary embodiment of polymeric amyloid fibers used to form strong macroscopic fibers including toughness of 16× amyloid fibers and recombinant silk fibers in accordance with the present disclosure. Error bars represent standard deviation. *p<0.05, two-tailed unpaired t-test. For the 16× amyloid fibers, n=10, and for the recombinant silk fibers, n=6.

FIG. 2A is an exemplary embodiment of a plasmid map used for expression of 16× KLVFFAE polymeric amyloid proteins in accordance with the present disclosure.

FIG. 2B is an exemplary embodiment of a plasmid map used for expression of 16× GDVIEV polymeric amyloid proteins in accordance with the present disclosure.

FIG. 2C is an exemplary embodiment of a plasmid map used for expression of 16× FGAILSS polymeric amyloid proteins in accordance with the present disclosure.

FIG. 2D is an exemplary embodiment of a plasmid map used for expression of 16× recombinant silk proteins in accordance with the present disclosure.

FIG. 3 is an exemplary embodiment of a Coomassie blue-stained 10% SDS-PAGE gel of Ni-NTA affinity chromatography purified 16× polymeric proteins in accordance with the present disclosure. Lane 1, molecular weight marker with their size labeled on the left. Lane 2, 16× KLVFFAE protein. Lane 3, 16× FGAILSS protein. Lane 4, 16× GDVIEV protein. Lane 5, 16× recombinant silk protein.

FIG. 4 is an exemplary embodiment of representative optical images of 16× fibers in accordance with the present disclosure. Scale bars (black lines) represent 20 μm.

FIG. 5 is an exemplary embodiment of representative SEM images of 16× polymeric amyloid fibers and recombinant silk fiber in accordance with the present disclosure. Cross sections were generated from fiber tensile testing. Scale bars in the images are 10 μm.

FIG. 6A is an exemplary embodiment of synchrotron-based wide-angle X-ray diffraction analyses of 16× KLVFFAE polymeric amyloid fibers in accordance with the present disclosure. Top panel, 1D radial intensity profile along the equator, with Gaussian fits for the (120) (dotted blue), (200) (dotted red), and three amorphous components (dotted green). Inset shows the 2D diffraction patterns. Middle panel, 1D intensity profile of the (120) peak as a function of azimuthal angle. Bottom panel, 1D intensity profile of the (200) peak as a function of azimuthal angle.

FIG. 6B is an exemplary embodiment of synchrotron-based wide-angle X-ray diffraction analyses of 16× GDVIEV polymeric amyloid fibers in accordance with the present disclosure. Top panel, 1D radial intensity profile along the equator, with Gaussian fits for the (120) (dotted blue), (200) (dotted red), and three amorphous components (dotted green). Inset shows the 2D diffraction patterns. Middle panel, 1D intensity profile of the (120) peak as a function of azimuthal angle. Bottom panel, 1D intensity profile of the (200) peak as a function of azimuthal angle.

FIG. 6C is an exemplary embodiment of synchrotron-based wide-angle X-ray diffraction analyses of 16× FGAILSS polymeric amyloid fibers in accordance with the present disclosure. Top panel, 1D radial intensity profile along the equator, with Gaussian fits for the (120) (dotted blue), (200) (dotted red), and three amorphous components (dotted green). Inset shows the 2D diffraction patterns. Middle panel, 1D intensity profile of the (120) peak as a function of azimuthal angle. Bottom panel, 1D intensity profile of the (200) peak as a function of azimuthal angle.

FIG. 6D is an exemplary embodiment of synchrotron-based wide-angle X-ray diffraction analyses of 16× recombinant silk fibers in accordance with the present disclosure. Top panel, 1D radial intensity profile along the equator, with Gaussian fits for the (120) (dotted blue), (200) (dotted red), and three amorphous components (dotted green). Inset shows the 2D diffraction patterns. Middle panel, 1D intensity profile of the (120) peak as a function of azimuthal angle. Bottom panel, 1D intensity profile of the (200) peak as a function of azimuthal angle.

FIG. 7A is an exemplary embodiment of a plasmid map used for expressing 48× FGAILSS proteins in accordance with the present disclosure.

FIG. 7B is an exemplary embodiment of a plasmid map used for expressing 96× FGAILSS proteins in accordance with the present disclosure.

FIG. 7C is an exemplary embodiment of a plasmid map used for expressing 128× FGAILSS proteins in accordance with the present disclosure.

FIG. 8 is an exemplary embodiment of production and characterization of 96× and 128× FGAILSS fibers including a Coomassie blue-stained 12% SDS-PAGE gel of E. coli whole cell lysate in accordance with the present disclosure. Lane 1, MW marker; lane 2, 96× FGAILSS protein before induction; lane 3, 96× FGAILSS protein after induction; lane 4, 128× FGAILSS protein before induction; lane 5, 128× FGAILSS protein after induction.

FIG. 9 is an exemplary embodiment of a Coomassie blue-stained 10% SDS-PAGE gel of purified FGAILSS polymeric proteins in accordance with the present disclosure. Lane 1, molecular weight marker with their size labeled on the left. Lane 2, 48× FGAILSS protein purified using Ni-NTA affinity chromatography. Lane 3, 96× FGAILSS protein purified using Ni-NTA affinity chromatography. Lane 4, 96× FGAILSS protein from Lane 3 was further purified using size-exclusion chromatography. Lane 5, 128× FGAILSS protein purified using Ni-NTA affinity chromatography. Lane 6, 128× FGAILSS protein from Lane 5 was further purified using size-exclusion chromatography.

FIG. 10A is an exemplary embodiment of production and characterization of 96× FGAILSS fibers including a representative stress-strain curve from tensile tests of 96× FGAILSS fibers in accordance with the present disclosure.

FIG. 10B is an exemplary embodiment of production and characterization of 128× FGAILSS fibers including a representative stress-strain curve from tensile tests of 128× FGAILSS fibers in accordance with the present disclosure.

FIG. 10C is an exemplary embodiment of production and characterization of 96× FGAILSS fibers including a representative SEM image of 96× FGAILSS fibers in accordance with the present disclosure. Scale bar in the image is 10 μm.

FIG. 10D is an exemplary embodiment of production and characterization of 128× FGAILSS fibers including a representative SEM image of 128× FGAILSS fibers in accordance with the present disclosure. Scale bar in the image is 10 μm.

FIG. 11A is an exemplary embodiment of SEC purification of 96× FGAILSS protein including a 10% SDS-PAGE gel of the elute protein fractions collected from SEC purification in accordance with the present disclosure. Lane 1, molecular weight marker with their size labeled on the left. Each of the rest of the lanes correspond to the part of spectrum labeled with the same number in FIG. 11B. The portions labeled in red were used for the preparation of protein dopes.

FIG. 11B is an exemplary embodiment of SEC purification of 96× FGAILSS protein including a SEC protein elution spectrum in accordance with the present disclosure. Proteins were detected by an UV detector at 280 nm. Each number corresponds to the same lane depicted in FIG. 11A. The portions labeled in red were used for the preparation of protein dopes.

FIG. 12A is an exemplary embodiment of SEC purification of 128× FGAILSS hybrid protein including a 10% SDS-PAGE gel of the elute protein fractions collected from SEC purification in accordance with the present disclosure. Lane 1, molecular weight marker with their size labeled on the left. Lane 2 and 3, elutions from Ni-NTA columns with 50 mM and 300 mM imidazole. Each of the rest of the lanes correspond to the part of the spectrum in FIG. 12B within the same column. The portions labeled in red were used for the preparation of protein dopes.

FIG. 12B is an exemplary embodiment of SEC purification of 128× FGAILSS hybrid protein including a SEC protein elution spectrum in accordance with the present disclosure. Proteins were detected by an UV detector at 280 nm. The portions labeled in red were used for the preparation of protein dopes.

FIG. 13A is an exemplary embodiment of mechanical properties of protein fibers including ultimate tensile stress of 16×-, 48×-, 96×-, and 128× FGAILSS fibers in accordance with the present disclosure. Error bars represent standard deviation. *p<0.05, two-tailed unpaired t-test. For the 16× and 96× fibers, n=10, for 48×, n=6, and for 128×, n=8.

FIG. 13B is an exemplary embodiment of mechanical properties of protein fibers including Young's Modulus of 16×-, 48×-, 96×-, and 128× FGAILSS fibers in accordance with the present disclosure. Error bars represent standard deviation. *p<0.05, two-tailed unpaired t-test. For the 16× and 96× fibers, n=10, for 48×, n=6, and for 128×, n=8.

FIG. 13C is an exemplary embodiment of mechanical properties of protein fibers including breaking strain of 16×-, 48×-, 96×-, and 128× FGAILSS fibers in accordance with the present disclosure. Error bars represent standard deviation. *p<0.05, two-tailed unpaired t-test. For the 16× and 96× fibers, n=10, for 48×, n=6, and for 128×, n=8.

FIG. 13D is an exemplary embodiment of mechanical properties of protein fibers including toughness of 16×-, 48×-, 96×-, and 128× FGAILSS fibers in accordance with the present disclosure. Error bars represent standard deviation. *p<0.05, two-tailed unpaired t-test. For the 16× and 96× fibers, n=10, for 48×, n=6, and for 128×, n=8.

FIG. 14 is an exemplary embodiment of representative stress-strain curves of 48× FGAILSS fibers in accordance with the present disclosure.

FIG. 15 is an exemplary embodiment of comparison of mechanical properties of protein fibers including a toughness-strength plot for various natural and synthetic protein-based fiber materials in accordance with the present disclosure. Black circles represent recombinant silk and fibers from previous work; brown circles represent natural dragline spider silk fibers; red star is from 128× FGAILSS of the present disclosure. “Synthetic 192×”—wet-spun post-translationally ligated 192-mer recombinant silk; “SRT-ELP Cys36×”—wet-spun recombinant chimeric protein with squid ring teeth segments, elastin-like polypeptide sequence and introduced cysteine residues, 36-mer; “rcSp2”—wet-spun recombinant silk with protein made from goat milk; “NT2RepCT”—biomimetic spinning of recombinant silk from protein with terminal regions; “MaSp1/MaSp2 4:1 mix”—Recombinant silk protein expressed and mixed before wet-spinning; “N1L(AQ)12NR3”-Biomimetic spinning of recombinant silk with engineered terminal regions. Data are listed in Table 10.

FIG. 16A is an exemplary embodiment of amide I Raman spectra for 16× GDVIEV fibers oriented parallel and perpendicular to the direction of laser polarization in accordance with the present disclosure.

FIG. 16B is an exemplary embodiment of amide I Raman spectra for 16× KLVFFAE fibers oriented parallel and perpendicular to the direction of laser polarization in accordance with the present disclosure.

FIG. 16C is an exemplary embodiment of amide I Raman spectra for 16× FGAILSS fibers oriented parallel and perpendicular to the direction of laser polarization in accordance with the present disclosure.

FIG. 16D is an exemplary embodiment of amide I Raman spectra for 16× recombinant silk fibers oriented parallel and perpendicular to the direction of laser polarization in accordance with the present disclosure.

### DETAILED DESCRIPTION OF THE DISCLOSURE

### Macroscopic Fibers Made of Polymeric Amyloid Proteins Display Gigapascal Tensile Strength

The ability of amyloid proteins to form stable β-sheet nanofibrils has made them potential candidates for material innovation in nanotechnology. However, such unique nano-scale features have rarely translated into attractive macroscopic properties for mechanically-demanding applications. Described herein are novel polymeric amyloid proteins formed by fusing numerous amyloid peptides (e.g., full amyloid peptides, amyloid peptide fragments, and combinations thereof) with flexible peptide sequences, such as flexible linkers from spidroin. The resulting polymeric amyloid proteins can be biosynthesized using engineered microbes and wet-spun into macroscopic fibers. Using this strategy, fibers from three different amyloid groups were fabricated. Structural analyses unveil the presence of β-nanocrystals that resemble the cross-β structure of amyloid nanofibrils. These polymeric amyloid fibers have displayed strong and molecular-weight-dependent mechanical properties. Fibers made of a protein polymer containing 128 repeats of the FGAILSS sequence displayed an average ultimate tensile strength of 0.98±0.08 GPa and an average toughness of 161±26 MJ/m³, surpassing most recombinant protein fibers and even some natural spider silk fibers. The design strategy and the biosynthetic approach described herein can be expanded to create numerous novel materials, and the macroscopic amyloid fibers enable a wide range of mechanically-demanding applications.

In some embodiments of the present disclosure, a system for synthesizing a recombinant polymeric amyloid in vivo is disclosed, the system comprising a host cell and a plasmid encoding tandem repeats of an amyloid peptide and a glycine-rich linker peptide.

In some embodiments of the present disclosure, a method for synthesizing a recombinant polymeric amyloid is disclosed, the method comprising synthesizing tandem repeats of an amyloid peptide and a glycine-rich linker peptide in vivo in a heterologous host.

In some embodiments of the present disclosure, a recombinant amyloid fiber is disclosed, the recombinant polymeric amyloid fiber comprising a plurality of polymeric amyloid fibrils each comprising a plurality of β-sheet crystals, wherein the β-sheet crystals comprise tandem repeats of an amyloid peptide and a glycine-rich linker peptide, and wherein the plurality of β-sheet crystals are aligned in parallel with the fiber axis.

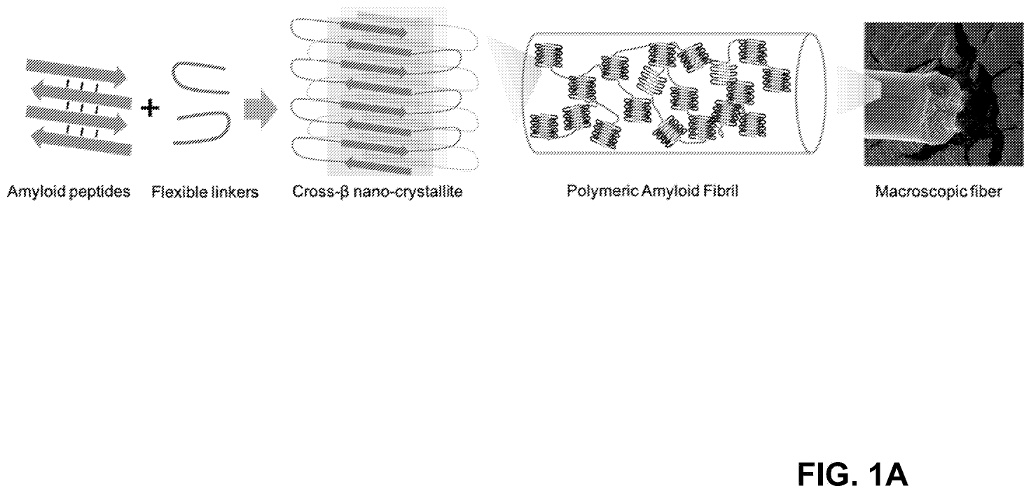

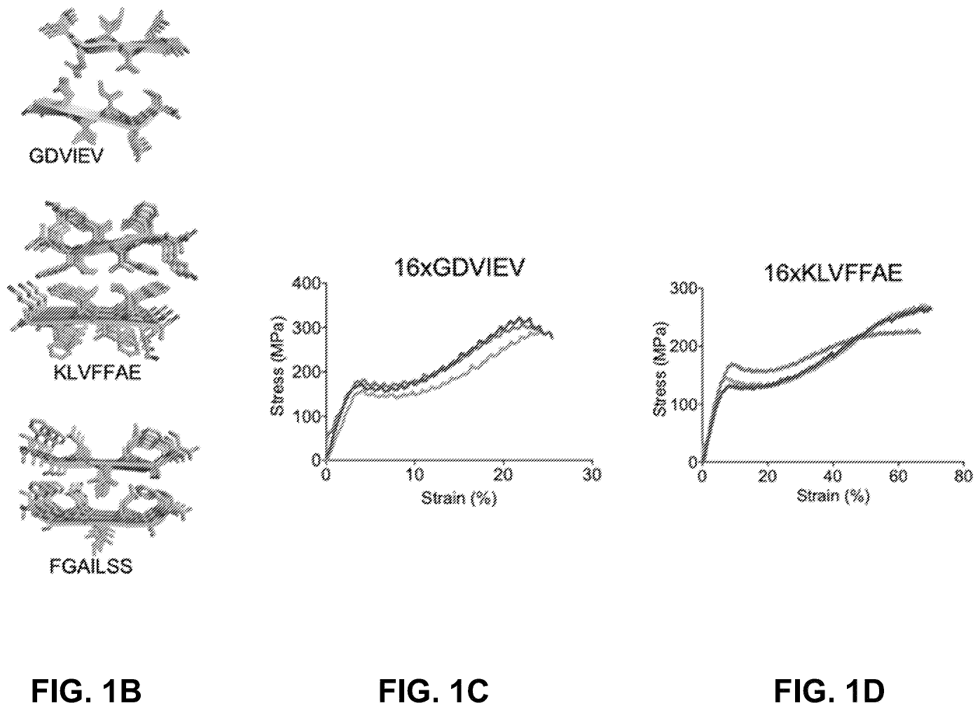

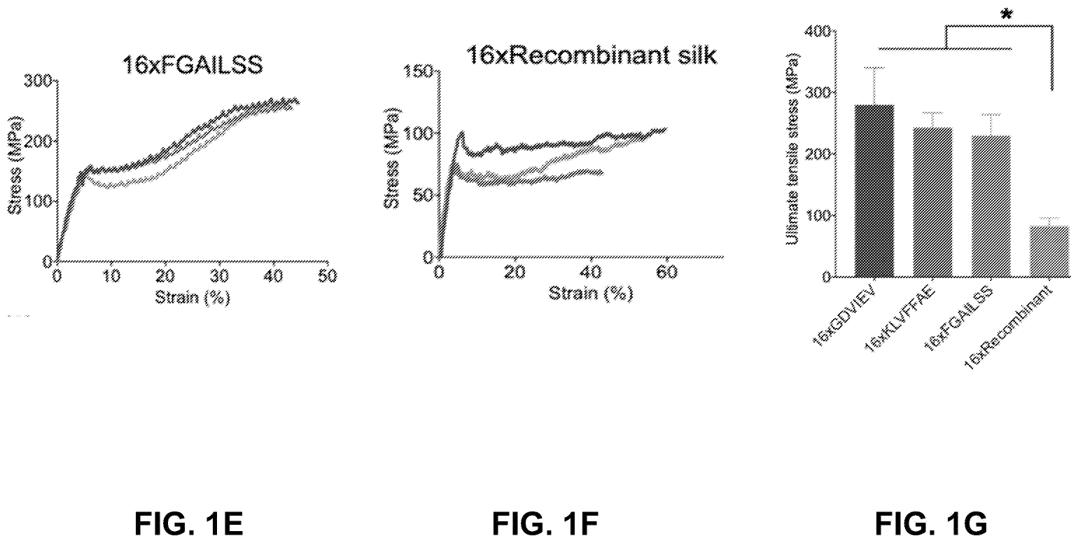

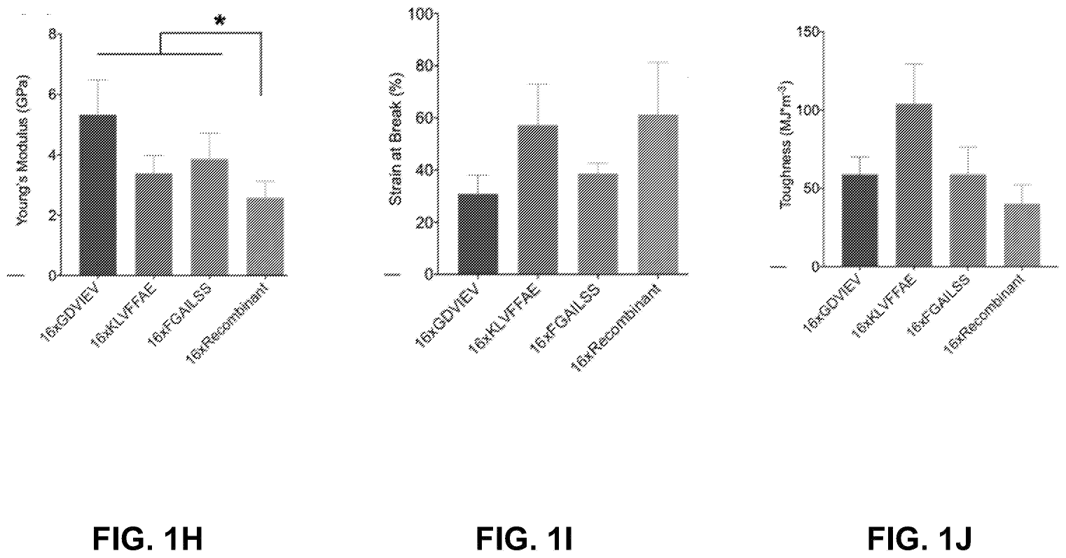

Described herein is the use of amyloid peptides in protein fibers, which due to the high β-sheet forming propensity of amyloids and the strong interaction within cross-β structures, in some embodiments promotes the formation of β crystals during spinning, leading to strong macroscopic fibers. This new type of fiber material comprises dozens or even hundreds of β-sheet-forming amyloid peptides connected by flexible peptide sequences, such as flexible glycine-rich peptide sequences of spidroins (see FIG. 1A).

The design principle was validated by creating polymeric amyloid fibers using amyloid peptides from the three distinct structural classes. Structural analyses using synchrotron-based wide-angle X-ray diffraction (WAXD) suggested greatly enhanced crystallinity compared to recombinant silk fibers of similar molecular weight spun from the same process. One of the polymeric amyloids FGAILSS was chosen to create high molecular weight variants. The 378 kDa 128× FGAILSS contains 128 repeats of FGAILSS peptide connected by glycine-rich linkers, and its fiber has displayed an ultimate tensile strength of 0.98±0.08 GPa and a toughness of 161±26 MJ/m⁻³, approaching the mechanical performance of some natural dragline spider silk fibers.

### Results

Different amyloids can assemble into different cross-β structures. An amyloid peptide was selected from each of three different structural classes: GDVIEV (SEQ ID NO: 1) represents a parallel amyloid, FGAILSS (SEQ ID NO: 3) represents an antiparallel homo-facial amyloid, and KLVFFAE (SEQ ID NO: 2) represents an antiparallel hetero-facial amyloid (see FIG. 1B, Table 1). A representative sequence from each category is exemplified herein. These results demonstrated that amyloid peptides from all three structural categories can be used to form strong fibers.

TABLE 1
Amyloid peptide sequences used herein.

| | Structural | PDB | |
|---|---|---|---|
| | Class | ID | Origin |
| GDVIEV | Class 4 | 3SGS | αB-crystallin |
| (SEQ ID NO: 1) | | | |
| KLVFFAE | Class 7 | 3OW9 | β-amyloid |
| (SEQ ID NO: 2) | | | |
| FGAILSS | Class 6 | 5E61 | islet amyloid |
| (SEQ ID NO: 3) | | | polypeptide |

The glycine-rich sequence from MaSp1 of Nephila clavipes spidroin was used to connect 16 of each amyloid peptide (see Table 2), generating a 16-mer for each amyloid (i.e., 16× GDV, 16× FGA, and 16× KLV, all approximately 50 kDa).

TABLE 2
Summary of amino acid sequences of
all proteins used herein.

| | 1x Sequence |
|---|---|
| 16xGDVIEV | MAKTKGTASGRGGLGGQGAGGDVIEVGGAGQGGYGGLG |
| (SEQ ID | SQGTSGRGGLGGQGAGGDVIEVGGAGQGGYGGLGSQGT |
| NO: 4) | SGRGGLGGQGAGGDVIEVGGAGQGGYGGLGSQGTSGRG |
| | GLGGQGAGGDVIEVGGAGQGGYGGLGSQGTSGRGGLGG |
| | QGAGGDVIEVGGAGQGGYGGLGSQGTSGRGGLGGQGAG |
| | GDVIEVGGAGQGGYGGLGSQGTSGRGGLGGQGAGGDVI |
| | EVGGAGQGGYGGLGSQGTSGRGGLGGQGAGGDVIEVGG |
| | AGQGGYGGLGSQGTSGRGGLGGQGAGGDVIEVGGAGQG |
| | GYGGLGSQGTSGRGGLGGQGAGGDVIEVGGAGQGGYGG |
| | LGSQGTSGRGGLGGQGAGGDVIEVGGAGQGGYGGLGSQ |
| | GTSGRGGLGGQGAGGDVIEVGGAGQGGYGGLGSQGTSG |
| | RGGLGGQGAGGDVIEVGGAGQGGYGGLGSQGTSGRGGL |
| | GGQGAGGDVIEVGGAGQGGYGGLGSQGTSGRGGLGGQG |
| | AGGDVIEVGGAGQGGYGGLGSQGTSGRGGLGGQGAGGD |
| | VIEVGGAGQGGYGGLGSQGTSSGHHHHHHHHHH |
| 16xKLVFFAE | MAKTKGTASGRGGLGGQGAGKLVFFAEGGAGQGGYGGL |
| (SEQ ID | GSQGTSGRGGLGGQGAGKLVFFAEGGAGQGGYGGLGSQ |
| NO: 5) | GTSGRGGLGGQGAGKLVFFAEGGAGQGGYGGLGSQGTS |
| | GRGGLGGQGAGKLVFFAEGGAGQGGYGGLGSQGTSGRG |
| | GLGGQGAGKLVFFAEGGAGQGGYGGLGSQGTSGRGGLG |
| | GQGAGKLVFFAEGGAGQGGYGGLGSQGTSGRGGLGGQG |
| | AGKLVFFAEGGAGQGGYGGLGSQGTSGRGGLGGQGAGK |
| | LVFFAEGGAGQGGYGGLGSQGTSGRGGLGGQGAGKLVF |
| | FAEGGAGQGGYGGLGSQGTSGRGGLGGQGAGKLVFFAE |
| | GGAGQGGYGGLGSQGTSGRGGLGGQGAGKLVFFAEGGA |
| | GQGGYGGLGSQGTSGRGGLGGQGAGKLVFFAEGGAGQG |
| | GYGGLGSQGTSGRGGLGGQGAGKLVFFAEGGAGQGGYG |
| | GLGSQGTSGRGGLGGQGAGKLVFFAEGGAGQGGYGGLG |
| | SQGTSGRGGLGGQGAGKLVFFAEGGAGQGGYGGLGSQG |
| | TSGRGGLGGQGAGKLVFFAEGGAGQGGYGGLGSQGTSS |
| | GHHHHHHHHHH |
| 16xFGAILSS | MAKTKGTASGRGGLGGQGAGFGAILSSGGAGQGGYGGL |
| (SEQ ID | GSQGTSGRGGLGGQGAGFGAILSSGGAGQGGYGGLGSQ |
| NO: 6) | GTSGRGGLGGQGAGFGAILSSGGAGQGGYGGLGSQGTS |
| | GRGGLGGQGAGFGAILSSGGAGQGGYGGLGSQGTSGRG |
| | GLGGQGAGFGAILSSGGAGQGGYGGLGSQGTSGRGGLG |
| | GQGAGFGAILSSGGAGQGGYGGLGSQGTSGRGGLGGQG |
| | AGFGAILSSGGAGQGGYGGLGSQGTSGRGGLGGQGAGF |
| | GAILSSGGAGQGGYGGLGSQGTSGRGGLGGQGAGFGAI |
| | LSSGGAGQGGYGGLGSQGTSGRGGLGGQGAGFGAILSS |
| | GGAGQGGYGGLGSQGTSGRGGLGGQGAGFGAILSSGGA |
| | GQGGYGGLGSQGTSGRGGLGGQGAGFGAILSSGGAGQG |
| | GYGGLGSQGTSGRGGLGGQGAGFGAILSSGGAGQGGYG |
| | GLGSQGTSGRGGLGGQGAGFGAILSSGGAGQGGYGGLG |
| | SQGTSGRGGLGGQGAGFGAILSSGGAGQGGYGGLGSQG |
| | TSGRGGLGGQGAGFGAILSSGGAGQGGYGGLGSQGTSS |
| | GHHHHHHHHHH |
| 16xRecomb- | MAKTKGTASGRGGLGGQGAGAAAAAGGAGQGGYGGLGS |
| inant | QGTSGRGGLGGQGAGAAAAAGGAGQGGYGGLGSQGTSG |
| silk (SEQ | RGGLGGQGAGAAAAAGGAGQGGYGGLGSQGTSGRGGLG |
| ID NO: 7) | GQGAGAAAAAGGAGQGGYGGLGSQGTSGRGGLGGQGAG |
| | AAAAAGGAGQGGYGGLGSQGTSGRGGLGGQGAGAAAAA |
| | GGAGQGGYGGLGSQGTSGRGGLGGQGAGAAAAAGGAGQ |
| | GGYGGLGSQGTSGRGGLGGQGAGAAAAAGGAGQGGYGG |
| | LGSQGTSGRGGLGGQGAGAAAAAGGAGQGGYGGLGSQG |
| | TSGRGGLGGQGAGAAAAAGGAGQGGYGGLGSQGTSGRG |
| | GLGGQGAGAAAAAGGAGQGGYGGLGSQGTSGRGGLGGQ |
| | GAGAAAAAGGAGQGGYGGLGSQGTSGRGGLGGQGAGAA |
| | AAAGGAGQGGYGGLGSQGTSGRGGLGGQGAGAAAAAGG |
| | AGQGGYGGLGSQGTSGRGGLGGQGAGAAAAAGGAGQGG |
| | YGGLGSQGTSGRGGLGGQGAGAAAAAGGAGQGGYGGLG |
| | SQGTSSGHHHHHHHHHH |
| 48xFGAILSS | MAKTKGTASGRGGLGGQGAGFGAILSSGGAGQGGYGGL |
| (SEQ ID | GSQGTSGRGGLGGQGAGFGAILSSGGAGQGGYGGLGSQ |
| NO: 8) | GTSGRGGLGGQGAGFGAILSSGGAGQGGYGGLGSQGTS |
| | GRGGLGGQGAGFGAILSSGGAGQGGYGGLGSQGTSGRG |
| | GLGGQGAGFGAILSSGGAGQGGYGGLGSQGTSGRGGLG |
| | GQGAGFGAILSSGGAGQGGYGGLGSQGTSGRGGLGGQG |
| | AGFGAILSSGGAGQGGYGGLGSQGTSGRGGLGGQGAGF |
| | GAILSSGGAGQGGYGGLGSQGTSGRGGLGGQGAGFGAI |
| | LSSGGAGQGGYGGLGSQGTSGRGGLGGQGAGFGAILSS |
| | GGAGQGGYGGLGSQGTSGRGGLGGQGAGFGAILSSGGA |
| | GQGGYGGLGSQGTSGRGGLGGQGAGFGAILSSGGAGQG |
| | GYGGLGSQGTSGRGGLGGQGAGFGAILSSGGAGQGGYG |
| | GLGSQGTSGRGGLGGQGAGFGAILSSGGAGQGGYGGLG |
| | SQGTSGRGGLGGQGAGFGAILSSGGAGQGGYGGLGSQG |
| | TSGRGGLGGQGAGFGAILSSGGAGQGGYGGLGSQGTSG |
| | RGGLGGQGAGFGAILSSGGAGQGGYGGLGSQGTSGRGG |
| | LGGQGAGFGAILSSGGAGQGGYGGLGSQGTSGRGGLGG |
| | QGAGFGAILSSGGAGQGGYGGLGSQGTSGRGGLGGQGA |
| | GFGAILSSGGAGQGGYGGLGSQGTSGRGGLGGQGAGFG |
| | AILSSGGAGQGGYGGLGSQGTSGRGGLGGQGAGFGAIL |
| | SSGGAGQGGYGGLGSQGTSGRGGLGGQGAGFGAILSSG |
| | GAGQGGYGGLGSQGTSGRGGLGGQGAGFGAILSSGGAG |
| | QGGYGGLGSQGTSGRGGLGGQGAGFGAILSSGGAGQGG |
| | YGGLGSQGTSGRGGLGGQGAGFGAILSSGGAGQGGYGG |
| | LGSQGTSGRGGLGGQGAGFGAILSSGGAGQGGYGGLGS |
| | QGTSGRGGLGGQGAGFGAILSSGGAGQGGYGGLGSQGT |
| | SGRGGLGGQGAGFGAILSSGGAGQGGYGGLGSQGTSGR |
| | GGLGGQGAGFGAILSSGGAGQGGYGGLGSQGTSGRGGL |
| | GGQGAGFGAILSSGGAGQGGYGGLGSQGTSGRGGLGGQ |
| | GAGFGAILSSGGAGQGGYGGLGSQGTSGRGGLGGQGAG |
| | FGAILSSGGAGQGGYGGLGSQGTSGRGGLGGQGAGFGA |
| | ILSSGGAGQGGYGGLGSQGTSGRGGLGGQGAGFGAILS |
| | SGGAGQGGYGGLGSQGTSGRGGLGGQGAGFGAILSSGG |
| | AGQGGYGGLGSQGTSGRGGLGGQGAGFGAILSSGGAGQ |
| | GGYGGLGSQGTSGRGGLGGQGAGFGAILSSGGAGQGGY |
| | GGLGSQGTSGRGGLGGQGAGFGAILSSGGAGQGGYGGL |
| | GSQGTSGRGGLGGQGAGFGAILSSGGAGQGGYGGLGSQ |
| | GTSGRGGLGGQGAGFGAILSSGGAGQGGYGGLGSQGTS |
| | GRGGLGGQGAGFGAILSSGGAGQGGYGGLGSQGTSGRG |
| | GLGGQGAGFGAILSSGGAGQGGYGGLGSQGTSGRGGLG |
| | GQGAGFGAILSSGGAGQGGYGGLGSQGTSGRGGLGGQG |
| | AGFGAILSSGGAGQGGYGGLGSQGTSGRGGLGGQGAGF |
| | GAILSSGGAGQGGYGGLGSQGTSGRGGLGGQGAGFGAI |
| | LSSGGAGQGGYGGLGSQGTSGRGGLGGQGAGFGAILSS |
| | GGAGQGGYGGLGSQGTSSGHHHHHHHHHH |
| 96xFGAILSS | MAKTKGTASGRGGLGGQGAGFGAILSSGGAGQGGYGGL |
| (SEQ ID | GSQGTSGRGGLGGQGAGFGAILSSGGAGQGGYGGLGSQ |
| NO: 9) | GTSGRGGLGGQGAGFGAILSSGGAGQGGYGGLGSQGTS |
| | GRGGLGGQGAGFGAILSSGGAGQGGYGGLGSQGTSGRG |
| | GLGGQGAGFGAILSSGGAGQGGYGGLGSQGTSGRGGLG |
| | GQGAGFGAILSSGGAGQGGYGGLGSQGTSGRGGLGGQG |
| | AGFGAILSSGGAGQGGYGGLGSQGTSGRGGLGGQGAGF |
| | GAILSSGGAGQGGYGGLGSQGTSGRGGLGGQGAGFGAI |
| | LSSGGAGQGGYGGLGSQGTSGRGGLGGQGAGFGAILSS |
| | GGAGQGGYGGLGSQGTSGRGGLGGQGAGFGAILSSGGA |
| | GQGGYGGLGSQGTSGRGGLGGQGAGFGAILSSGGAGQG |
| | GYGGLGSQGTSGRGGLGGQGAGFGAILSSGGAGQGGYG |
| | GLGSQGTSGRGGLGGQGAGFGAILSSGGAGQGGYGGLG |
| | SQGTSGRGGLGGQGAGFGAILSSGGAGQGGYGGLGSQG |
| | TSGRGGLGGQGAGFGAILSSGGAGQGGYGGLGSQGTSG |
| | RGGLGGQGAGFGAILSSGGAGQGGYGGLGSQGTSGRGG |
| | LGGQGAGFGAILSSGGAGQGGYGGLGSQGTSGRGGLGG |
| | QGAGFGAILSSGGAGQGGYGGLGSQGTSGRGGLGGQGA |
| | GFGAILSSGGAGQGGYGGLGSQGTSGRGGLGGQGAGFG |
| | AILSSGGAGQGGYGGLGSQGTSGRGGLGGQGAGFGAIL |
| | SSGGAGQGGYGGLGSQGTSGRGGLGGQGAGFGAILSSG |
| | GAGQGGYGGLGSQGTSGRGGLGGQGAGFGAILSSGGAG |
| | QGGYGGLGSQGTSGRGGLGGQGAGFGAILSSGGAGQGG |
| | YGGLGSQGTSGRGGLGGQGAGFGAILSSGGAGQGGYGG |
| | LGSQGTSGRGGLGGQGAGFGAILSSGGAGQGGYGGLGS |
| | QGTSGRGGLGGQGAGFGAILSSGGAGQGGYGGLGSQGT |
| | SGRGGLGGQGAGFGAILSSGGAGQGGYGGLGSQGTSGR |
| | GGLGGQGAGFGAILSSGGAGQGGYGGLGSQGTSGRGGL |
| | GGQGAGFGAILSSGGAGQGGYGGLGSQGTSGRGGLGGQ |
| | GAGFGAILSSGGAGQGGYGGLGSQGTSGRGGLGGQGAG |
| | FGAILSSGGAGQGGYGGLGSQGTSGRGGLGGQGAGFGA |
| | ILSSGGAGQGGYGGLGSQGTSGRGGLGGQGAGFGAILS |
| | SGGAGQGGYGGLGSQGTSGRGGLGGQGAGFGAILSSGG |
| | AGQGGYGGLGSQGTSGRGGLGGQGAGFGAILSSGGAGQ |
| | GGYGGLGSQGTSGRGGLGGQGAGFGAILSSGGAGQGGY |
| | GGLGSQGTSGRGGLGGQGAGFGAILSSGGAGQGGYGGL |
| | GSQGTSGRGGLGGQGAGFGAILSSGGAGQGGYGGLGSQ |
| | GTSGRGGLGGQGAGFGAILSSGGAGQGGYGGLGSQGTS |
| | GRGGLGGQGAGFGAILSSGGAGQGGYGGLGSQGTSGRG |
| | GLGGQGAGFGAILSSGGAGQGGYGGLGSQGTSGRGGLG |
| | GQGAGFGAILSSGGAGQGGYGGLGSQGTSGRGGLGGQG |
| | AGFGAILSSGGAGQGGYGGLGSQGTSGRGGLGGQGAGF |
| | GAILSSGGAGQGGYGGLGSQGTSGRGGLGGQGAGFGAI |
| | LSSGGAGQGGYGGLGSQGTSGRGGLGGQGAGFGAILSS |
| | GGAGQGGYGGLGSQGTSGRGGLGGQGAGFGAILSSGGA |
| | GQGGYGGLGSQGTSGRGGLGGQGAGFGAILSSGGAGQG |
| | GYGGLGSQGTSGRGGLGGQGAGFGAILSSGGAGQGGYG |
| | GLGSQGTSGRGGLGGQGAGFGAILSSGGAGQGGYGGLG |
| | SQGTSGRGGLGGQGAGFGAILSSGGAGQGGYGGLGSQG |
| | TSGRGGLGGQGAGFGAILSSGGAGQGGYGGLGSQGTSG |
| | RGGLGGQGAGFGAILSSGGAGQGGYGGLGSQGTSGRGG |
| | LGGQGAGFGAILSSGGAGQGGYGGLGSQGTSGRGGLGG |
| | QGAGFGAILSSGGAGQGGYGGLGSQGTSGRGGLGGQGA |
| | GFGAILSSGGAGQGGYGGLGSQGTSGRGGLGGQGAGFG |
| | AILSSGGAGQGGYGGLGSQGTSGRGGLGGQGAGFGAIL |
| | SSGGAGQGGYGGLGSQGTSGRGGLGGQGAGFGAILSSG |
| | GAGQGGYGGLGSQGTSGRGGLGGQGAGFGAILSSGGAG |
| | QGGYGGLGSQGTSGRGGLGGQGAGFGAILSSGGAGQGG |
| | YGGLGSQGTSGRGGLGGQGAGFGAILSSGGAGQGGYGG |
| | LGSQGTSGRGGLGGQGAGFGAILSSGGAGQGGYGGLGS |
| | QGTSGRGGLGGQGAGFGAILSSGGAGQGGYGGLGSQGT |
| | SGRGGLGGQGAGFGAILSSGGAGQGGYGGLGSQGTSGR |
| | GGLGGQGAGFGAILSSGGAGQGGYGGLGSQGTSGRGGL |
| | GGQGAGFGAILSSGGAGQGGYGGLGSQGTSGRGGLGGQ |
| | GAGFGAILSSGGAGQGGYGGLGSQGTSGRGGLGGQGAG |
| | FGAILSSGGAGQGGYGGLGSQGTSGRGGLGGQGAGFGA |
| | ILSSGGAGQGGYGGLGSQGTSGRGGLGGQGAGFGAILS |
| | SGGAGQGGYGGLGSQGTSGRGGLGGQGAGFGAILSSGG |
| | AGQGGYGGLGSQGTSGRGGLGGQGAGFGAILSSGGAGQ |
| | GGYGGLGSQGTSGRGGLGGQGAGFGAILSSGGAGQGGY |
| | GGLGSQGTSGRGGLGGQGAGFGAILSSGGAGQGGYGGL |
| | GSQGTSGRGGLGGQGAGFGAILSSGGAGQGGYGGLGSQ |
| | GTSGRGGLGGQGAGFGAILSSGGAGQGGYGGLGSQGTS |
| | GRGGLGGQGAGFGAILSSGGAGQGGYGGLGSQGTSGRG |
| | GLGGQGAGFGAILSSGGAGQGGYGGLGSQGTSGRGGLG |
| | GQGAGFGAILSSGGAGQGGYGGLGSQGTSGRGGLGGQG |
| | AGFGAILSSGGAGQGGYGGLGSQGTSGRGGLGGQGAGF |
| | GAILSSGGAGQGGYGGLGSQGTSGRGGLGGQGAGFGAI |
| | LSSGGAGQGGYGGLGSQGTSGRGGLGGQGAGFGAILSS |
| | GGAGQGGYGGLGSQGTSGRGGLGGQGAGFGAILSSGGA |
| | GQGGYGGLGSQGTSGRGGLGGQGAGFGAILSSGGAGQG |
| | GYGGLGSQGTSGRGGLGGQGAGFGAILSSGGAGQGGYG |
| | GLGSQGTSGRGGLGGQGAGFGAILSSGGAGQGGYGGLG |
| | SQGTSGRGGLGGQGAGFGAILSSGGAGQGGYGGLGSQG |
| | TSGRGGLGGQGAGFGAILSSGGAGQGGYGGLGSQGTSG |
| | RGGLGGQGAGFGAILSSGGAGQGGYGGLGSQGTSGRGG |
| | LGGQGAGFGAILSSGGAGQGGYGGLGSQGTSGRGGLGG |
| | QGAGFGAILSSGGAGQGGYGGLGSQGTSGRGGLGGQGA |
| | GFGAILSSGGAGQGGYGGLGSQGTSSGHHHHHHHHHH |
| 128x | MAKTKGTASGRGGLGGQGAGFGAILSSGGAGQGGYGGL |
| FGAILSS | GSQGTSGRGGLGGQGAGFGAILSSGGAGQGGYGGLGSQ |
| (SEQ ID | GTSGRGGLGGQGAGFGAILSSGGAGQGGYGGLGSQGTS |
| NO: 10) | GRGGLGGQGAGFGAILSSGGAGQGGYGGLGSQGTSGRG |
| | GLGGQGAGFGAILSSGGAGQGGYGGLGSQGTSGRGGLG |
| | GQGAGFGAILSSGGAGQGGYGGLGSQGTSGRGGLGGQG |
| | AGFGAILSSGGAGQGGYGGLGSQGTSGRGGLGGQGAGF |
| | GAILSSGGAGQGGYGGLGSQGTSGRGGLGGQGAGFGAI |
| | LSSGGAGQGGYGGLGSQGTSGRGGLGGQGAGFGAILSS |
| | GGAGQGGYGGLGSQGTSGRGGLGGQGAGFGAILSSGGA |
| | GQGGYGGLGSQGTSGRGGLGGQGAGFGAILSSGGAGQG |
| | GYGGLGSQGTSGRGGLGGQGAGFGAILSSGGAGQGGYG |
| | GLGSQGTSGRGGLGGQGAGFGAILSSGGAGQGGYGGLG |
| | SQGTSGRGGLGGQGAGFGAILSSGGAGQGGYGGLGSQG |
| | TSGRGGLGGQGAGFGAILSSGGAGQGGYGGLGSQGTSG |
| | RGGLGGQGAGFGAILSSGGAGQGGYGGLGSQGTSGRGG |
| | LGGQGAGFGAILSSGGAGQGGYGGLGSQGTSGRGGLGG |
| | QGAGFGAILSSGGAGQGGYGGLGSQGTSGRGGLGGQGA |
| | GFGAILSSGGAGQGGYGGLGSQGTSGRGGLGGQGAGFG |
| | AILSSGGAGQGGYGGLGSQGTSGRGGLGGQGAGFGAIL |
| | SSGGAGQGGYGGLGSQGTSGRGGLGGQGAGFGAILSSG |
| | GAGQGGYGGLGSQGTSGRGGLGGQGAGFGAILSSGGAG |
| | QGGYGGLGSQGTSGRGGLGGQGAGFGAILSSGGAGQGG |
| | YGGLGSQGTSGRGGLGGQGAGFGAILSSGGAGQGGYGG |
| | LGSQGTSGRGGLGGQGAGFGAILSSGGAGQGGYGGLGS |
| | QGTSGRGGLGGQGAGFGAILSSGGAGQGGYGGLGSQGT |
| | SGRGGLGGQGAGFGAILSSGGAGQGGYGGLGSQGTSGR |
| | GGLGGQGAGFGAILSSGGAGQGGYGGLGSQGTSGRGGL |
| | GGQGAGFGAILSSGGAGQGGYGGLGSQGTSGRGGLGGQ |
| | GAGFGAILSSGGAGQGGYGGLGSQGTSGRGGLGGQGAG |
| | FGAILSSGGAGQGGYGGLGSQGTSGRGGLGGQGAGFGA |
| | ILSSGGAGQGGYGGLGSQGTSGRGGLGGQGAGFGAILS |
| | SGGAGQGGYGGLGSQGTSGRGGLGGQGAGFGAILSSGG |
| | AGQGGYGGLGSQGTSGRGGLGGQGAGFGAILSSGGAGQ |
| | GGYGGLGSQGTSGRGGLGGQGAGFGAILSSGGAGQGGY |
| | GGLGSQGTSGRGGLGGQGAGFGAILSSGGAGQGGYGGL |
| | GSQGTSGRGGLGGQGAGFGAILSSGGAGQGGYGGLGSQ |
| | GTSGRGGLGGQGAGFGAILSSGGAGQGGYGGLGSQGTS |
| | GRGGLGGQGAGFGAILSSGGAGQGGYGGLGSQGTSGRG |
| | GLGGQGAGFGAILSSGGAGQGGYGGLGSQGTSGRGGLG |
| | GQGAGFGAILSSGGAGQGGYGGLGSQGTSGRGGLGGQG |
| | AGFGAILSSGGAGQGGYGGLGSQGTSGRGGLGGQGAGF |
| | GAILSSGGAGQGGYGGLGSQGTSGRGGLGGQGAGFGAI |
| | LSSGGAGQGGYGGLGSQGTSGRGGLGGQGAGFGAILSS |
| | GGAGQGGYGGLGSQGTSGRGGLGGQGAGFGAILSSGGA |
| | GQGGYGGLGSQGTSGRGGLGGQGAGFGAILSSGGAGQG |
| | GYGGLGSQGTSGRGGLGGQGAGFGAILSSGGAGQGGYG |
| | GLGSQGTSGRGGLGGQGAGFGAILSSGGAGQGGYGGLG |
| | SQGTSGRGGLGGQGAGFGAILSSGGAGQGGYGGLGSQG |
| | TSGRGGLGGQGAGFGAILSSGGAGQGGYGGLGSQGTSG |
| | RGGLGGQGAGFGAILSSGGAGQGGYGGLGSQGTSGRGG |
| | LGGQGAGFGAILSSGGAGQGGYGGLGSQGTSGRGGLGG |
| | QGAGFGAILSSGGAGQGGYGGLGSQGTSGRGGLGGQGA |
| | GFGAILSSGGAGQGGYGGLGSQGTSGRGGLGGQGAGFG |
| | AILSSGGAGQGGYGGLGSQGTSGRGGLGGQGAGFGAIL |
| | SSGGAGQGGYGGLGSQGTSGRGGLGGQGAGFGAILSSG |
| | GAGQGGYGGLGSQGTSGRGGLGGQGAGFGAILSSGGAG |
| | QGGYGGLGSQGTSGRGGLGGQGAGFGAILSSGGAGQGG |
| | YGGLGSQGTSGRGGLGGQGAGFGAILSSGGAGQGGYGG |
| | LGSQGTSGRGGLGGQGAGFGAILSSGGAGQGGYGGLGS |
| | QGTSGRGGLGGQGAGFGAILSSGGAGQGGYGGLGSQGT |
| | SGRGGLGGQGAGFGAILSSGGAGQGGYGGLGSQGTSGR |
| | GGLGGQGAGFGAILSSGGAGQGGYGGLGSQGTSGRGGL |
| | GGQGAGFGAILSSGGAGQGGYGGLGSQGTSGRGGLGGQ |
| | GAGFGAILSSGGAGQGGYGGLGSQGTSGRGGLGGQGAG |
| | FGAILSSGGAGQGGYGGLGSQGTSGRGGLGGQGAGFGA |
| | ILSSGGAGQGGYGGLGSQGTSGRGGLGGQGAGFGAILS |
| | SGGAGQGGYGGLGSQGTSGRGGLGGQGAGFGAILSSGG |
| | AGQGGYGGLGSQGTSGRGGLGGQGAGFGAILSSGGAGQ |
| | GGYGGLGSQGTSGRGGLGGQGAGFGAILSSGGAGQGGY |
| | GGLGSQGTSGRGGLGGQGAGFGAILSSGGAGQGGYGGL |
| | GSQGTSGRGGLGGQGAGFGAILSSGGAGQGGYGGLGSQ |
| | GTSGRGGLGGQGAGFGAILSSGGAGQGGYGGLGSQGTS |
| | GRGGLGGQGAGFGAILSSGGAGQGGYGGLGSQGTSGRG |
| | GLGGQGAGFGAILSSGGAGQGGYGGLGSQGTSGRGGLG |
| | GQGAGFGAILSSGGAGQGGYGGLGSQGTSGRGGLGGQG |
| | AGFGAILSSGGAGQGGYGGLGSQGTSGRGGLGGQGAGF |
| | GAILSSGGAGQGGYGGLGSQGTSGRGGLGGQGAGFGAI |
| | LSSGGAGQGGYGGLGSQGTSGRGGLGGQGAGFGAILSS |
| | GGAGQGGYGGLGSQGTSGRGGLGGQGAGFGAILSSGGA |
| | GQGGYGGLGSQGTSGRGGLGGQGAGFGAILSSGGAGQG |
| | GYGGLGSQGTSGRGGLGGQGAGFGAILSSGGAGQGGYG |
| | GLGSQGTSGRGGLGGQGAGFGAILSSGGAGQGGYGGLG |
| | SQGTSGRGGLGGQGAGFGAILSSGGAGQGGYGGLGSQG |
| | TSGRGGLGGQGAGFGAILSSGGAGQGGYGGLGSQGTSG |
| | RGGLGGQGAGFGAILSSGGAGQGGYGGLGSQGTSGRGG |
| | LGGQGAGFGAILSSGGAGQGGYGGLGSQGTSGRGGLGG |
| | QGAGFGAILSSGGAGQGGYGGLGSQGTSGRGGLGGQGA |
| | GFGAILSSGGAGQGGYGGLGSQGTSGRGGLGGQGAGFG |
| | AILSSGGAGQGGYGGLGSQGTSGRGGLGGQGAGFGAIL |
| | SSGGAGQGGYGGLGSQGTSGRGGLGGQGAGFGAILSSG |
| | GAGQGGYGGLGSQGTSGRGGLGGQGAGFGAILSSGGAG |
| | QGGYGGLGSQGTSGRGGLGGQGAGFGAILSSGGAGQGG |
| | YGGLGSQGTSGRGGLGGQGAGFGAILSSGGAGQGGYGG |
| | LGSQGTSGRGGLGGQGAGFGAILSSGGAGQGGYGGLGS |
| | QGTSGRGGLGGQGAGFGAILSSGGAGQGGYGGLGSQGT |
| | SGRGGLGGQGAGFGAILSSGGAGQGGYGGLGSQGTSGR |
| | GGLGGQGAGFGAILSSGGAGQGGYGGLGSQGTSGRGGL |
| | GGQGAGFGAILSSGGAGQGGYGGLGSQGTSGRGGLGGQ |
| | GAGFGAILSSGGAGQGGYGGLGSQGTSGRGGLGGQGAG |
| | FGAILSSGGAGQGGYGGLGSQGTSGRGGLGGQGAGFGA |
| | ILSSGGAGQGGYGGLGSQGTSGRGGLGGQGAGFGAILS |
| | SGGAGQGGYGGLGSQGTSGRGGLGGQGAGFGAILSSGG |
| | AGQGGYGGLGSQGTSGRGGLGGQGAGFGAILSSGGAGQ |
| | GGYGGLGSQGTSGRGGLGGQGAGFGAILSSGGAGQGGY |
| | GGLGSQGTSGRGGLGGQGAGFGAILSSGGAGQGGYGGL |
| | GSQGTSGRGGLGGQGAGFGAILSSGGAGQGGYGGLGSQ |
| | GTSGRGGLGGQGAGFGAILSSGGAGQGGYGGLGSQGTS |
| | GRGGLGGQGAGFGAILSSGGAGQGGYGGLGSQGTSGRG |
| | GLGGQGAGFGAILSSGGAGQGGYGGLGSQGTSGRGGLG |
| | GQGAGFGAILSSGGAGQGGYGGLGSQGTSGRGGLGGQG |
| | AGFGAILSSGGAGQGGYGGLGSQGTSGRGGLGGQGAGF |
| | GAILSSGGAGQGGYGGLGSQGTSGRGGLGGQGAGFGAI |
| | LSSGGAGQGGYGGLGSQGTSGRGGLGGQGAGFGAILSS |
| | GGAGQGGYGGLGSQGTSGRGGLGGQGAGFGAILSSGGA |
| | GQGGYGGLGSQGTSGRGGLGGQGAGFGAILSSGGAGQG |
| | GYGGLGSQGTSGRGGLGGQGAGFGAILSSGGAGQGGYG |
| | GLGSQGTSGRGGLGGQGAGFGAILSSGGAGQGGYGGLG |
| | SQGTSSGHHHHHHHHHH |

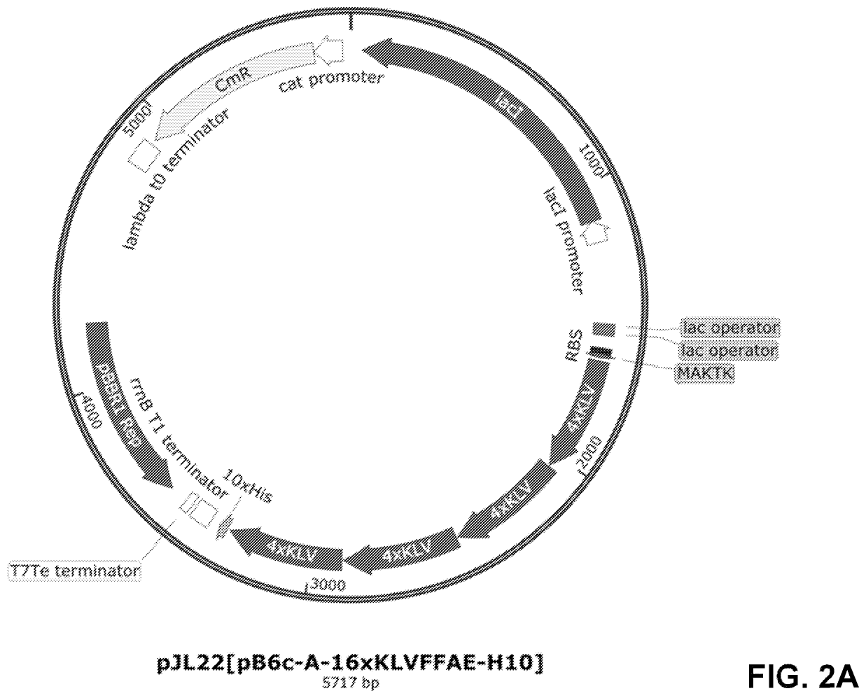

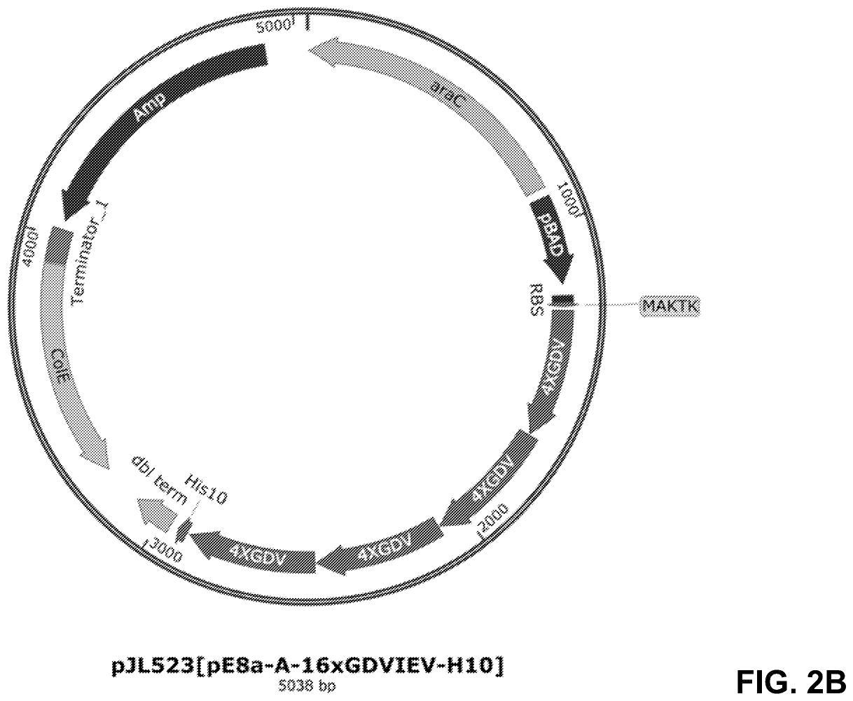

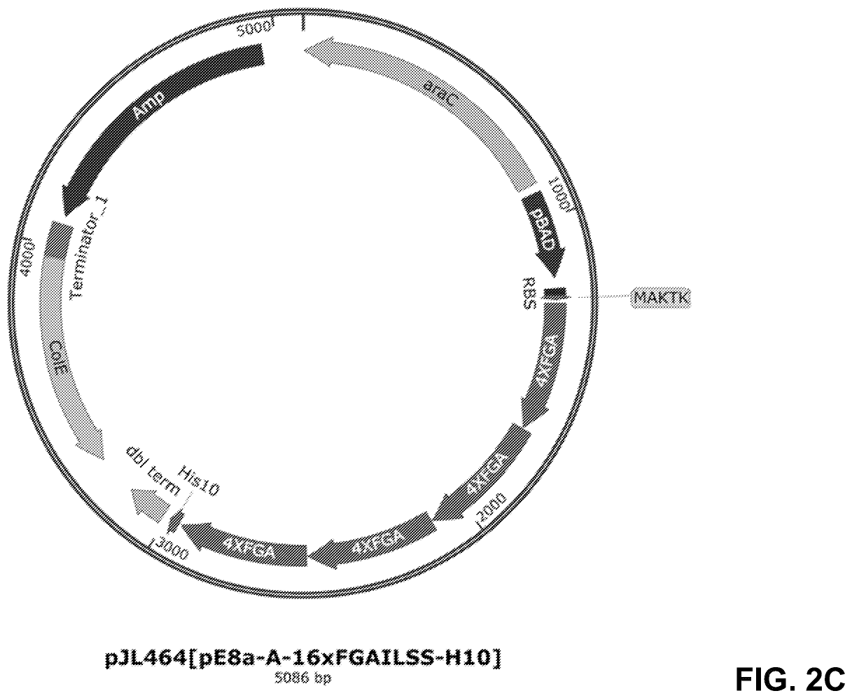

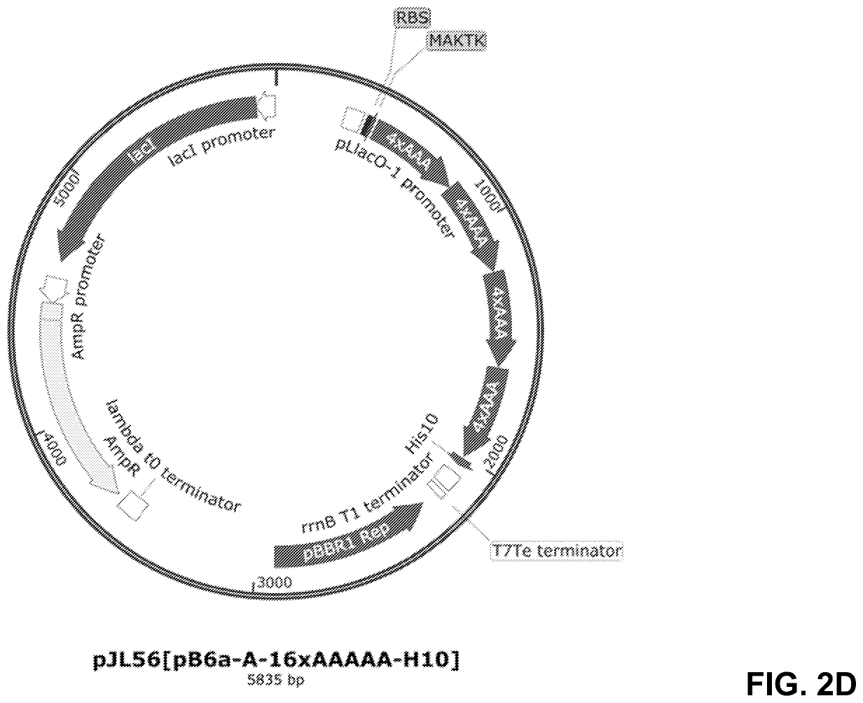

For comparison, a recombinant spider silk protein containing hexa-alanine instead of the amyloid peptide was also created (see FIG. 2A-FIG. 2D).

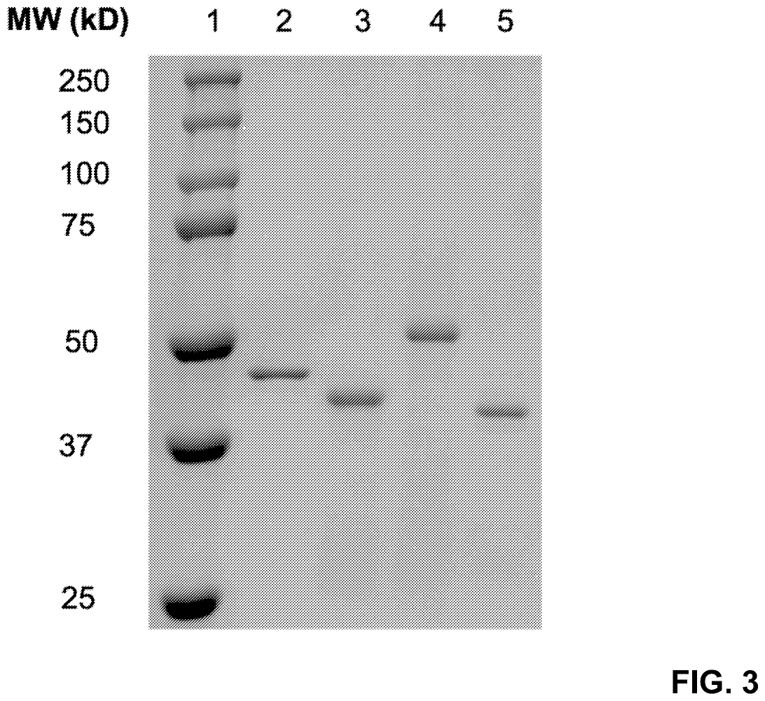

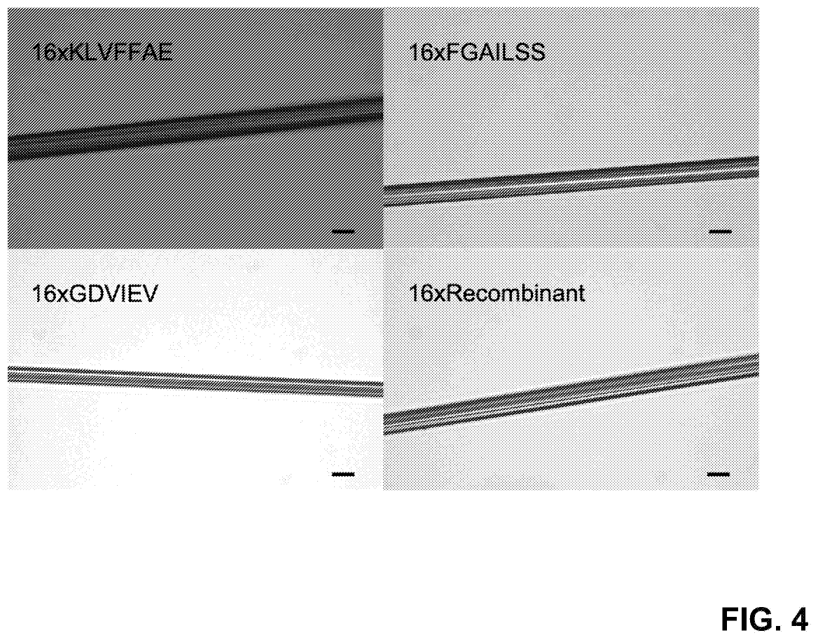

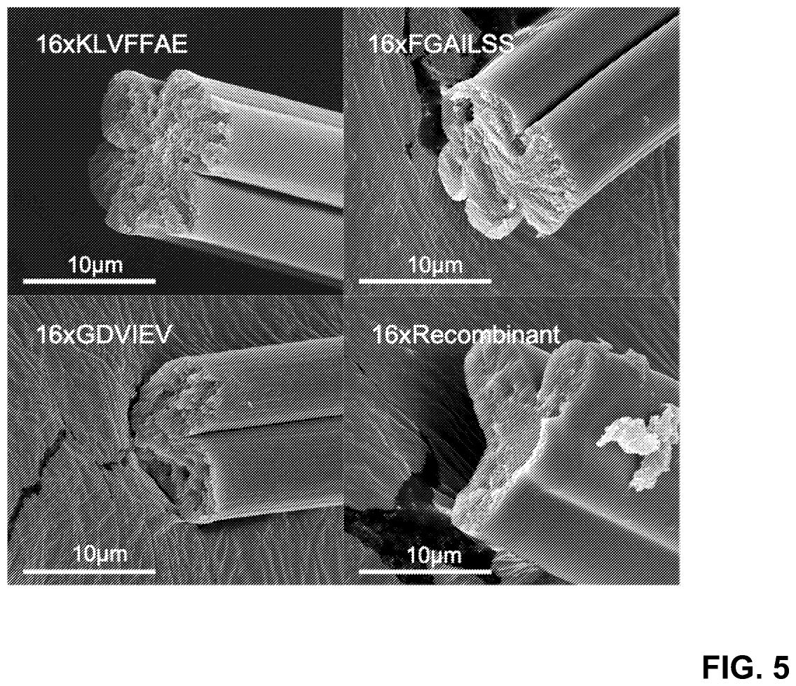

All proteins were expressed from synthetic DNA optimized to reduce repetitiveness in their coding sequences and purified with Ni-NTA affinity chromatography (see FIG. 3). Spinning dopes were prepared from 1,1,1,3,3,3-hexafluoro-2-propanol (HFIP) solution of purified proteins. Fibers were spun using standard wet-spinning techniques developed for recombinant spider silks. Optical microscopy and scanning electron microscopy (SEM) revealed that all fibers had smooth surfaces along the fiber axis (see FIG. 4 and FIG. 5).

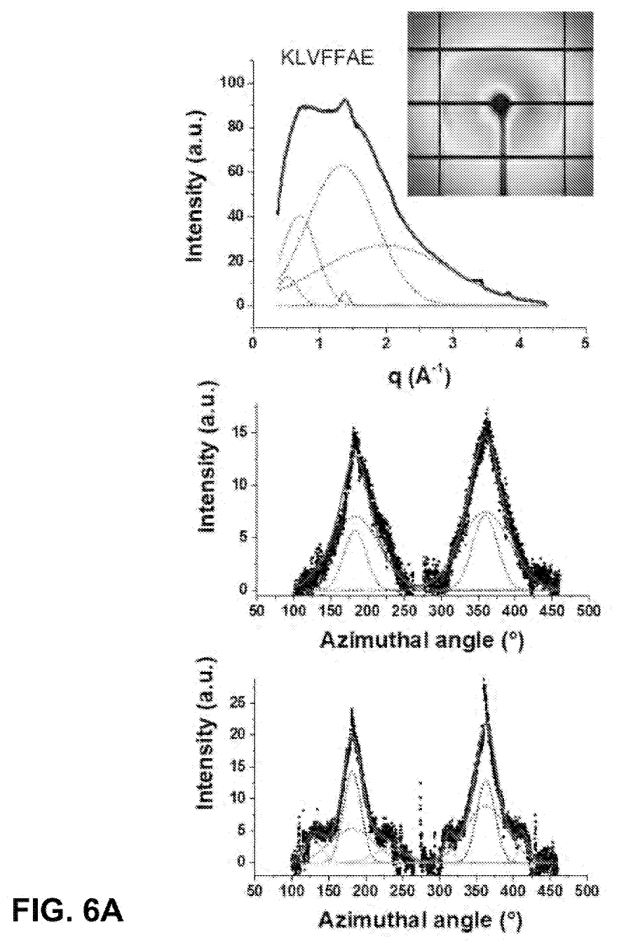

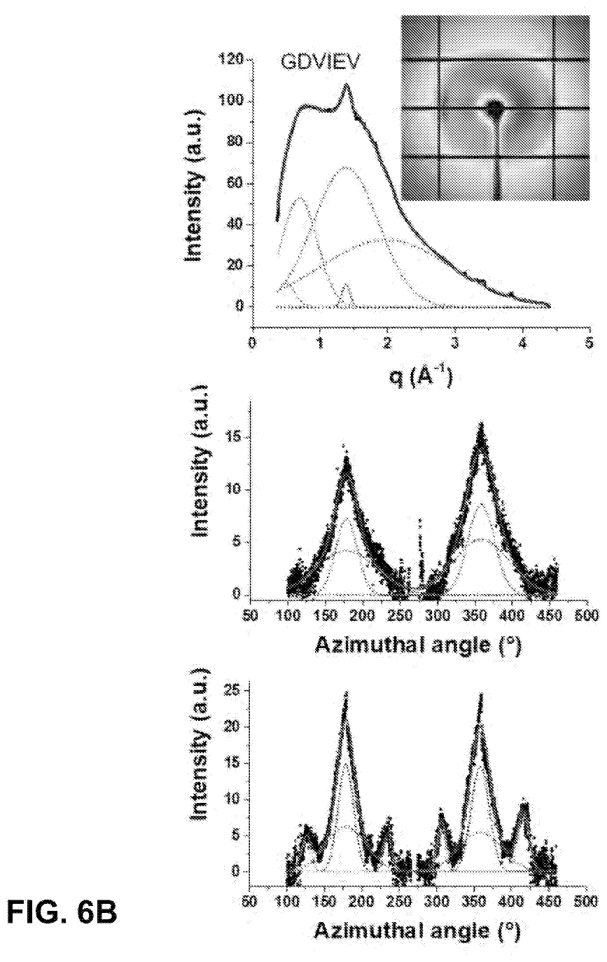

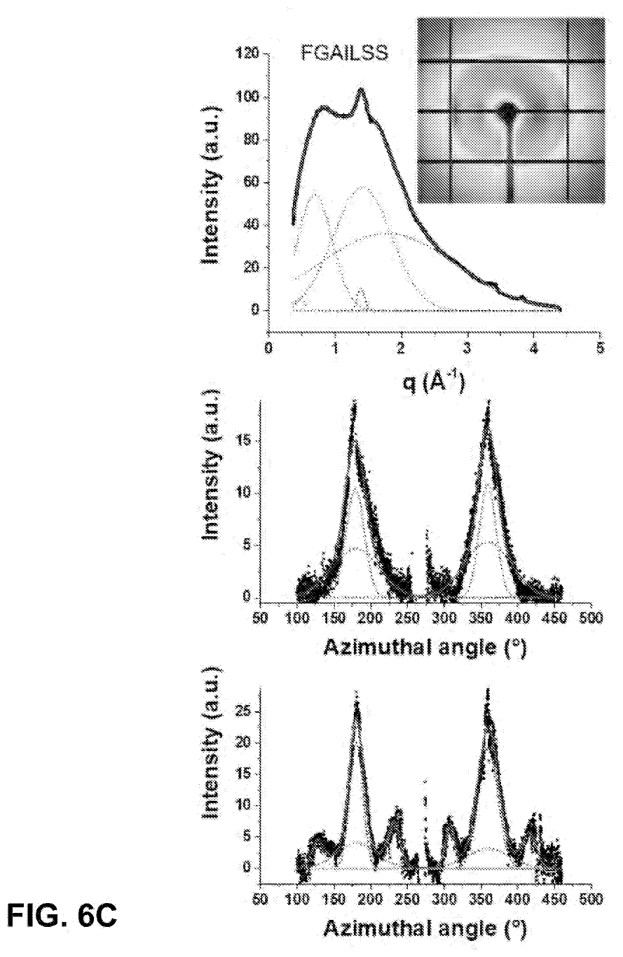

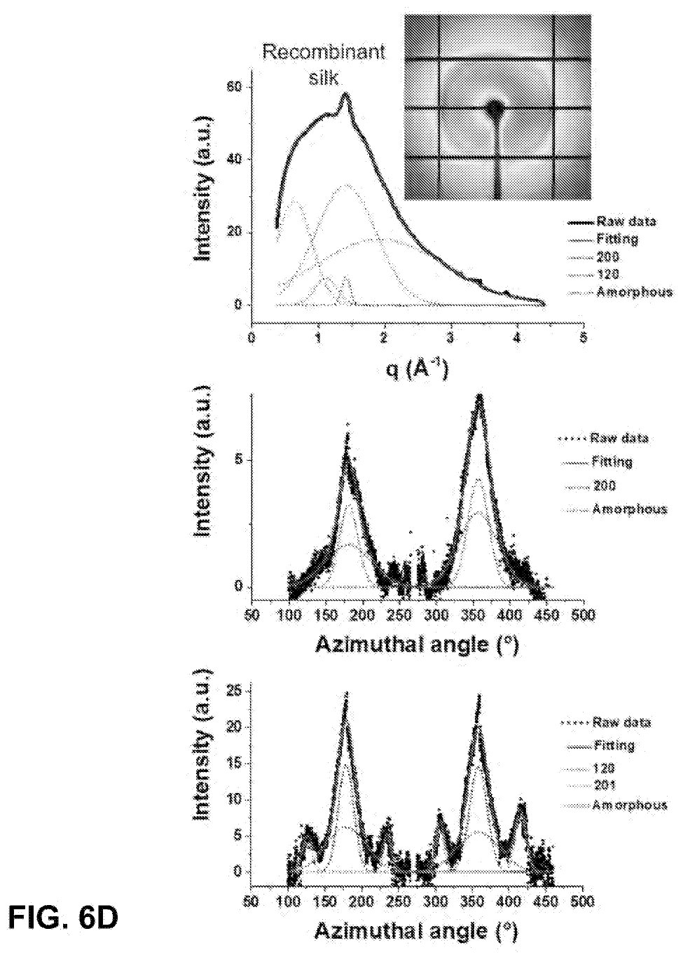

Standard tensile tests were performed on all 16× fibers (see FIG. 6A-FIG. 6D, Table 3-Table 6).

TABLE 3
Summary of mechanical properties of
16xKLVFFAE proteins disclosed herein.

| | Peak | | Strain | | |
|---|---|---|---|---|---|
| | stress/ | Modulus/ | at break/ | Toughness/ | Diameter/ |
| | MPa | GPa | % | MJ*m⁻³ | μm |
| KLV-1 | 240 | 4.0 | 38 | 80 | 14 |
| KLV-2 | 228 | 3.5 | 63 | 120 | 20 |
| KLV-3 | 245 | 3.8 | 49 | 90 | 20 |
| KLV-4 | 239 | 3.7 | 37 | 70 | 19 |
| KLV-5 | 188 | 3.6 | 50 | 80 | 22 |
| KLV-6 | 234 | 2.5 | 49 | 90 | 20 |
| KLV-7 | 265 | 2.8 | 62 | 120 | 17 |
| KLV-8 | 267 | 2.5 | 69 | 120 | 17 |
| KLV-9 | 248 | 4.1 | 88 | 150 | 17 |
| KLV-10 | 272 | 3.3 | 67 | 120 | 17 |
| Average | 243 | 3.4 | 57 | 104 | 18 |
| Standard | 24 | 0.6 | 16 | 26 | 2.3 |
| Deviation | | | | | |

TABLE 4
Summary of mechanical properties of 16x
FGAILSS proteins disclosed herein.

| | Peak | | Strain | | |
|---|---|---|---|---|---|
| | stress/ | Modulus/ | at break/ | Toughness/ | Diameter/ |
| | MPa | GPa | % | MJ*m⁻³ | μm |
| FGA-1 | 202 | 3.9 | 34 | 40 | 17 |
| FGA-2 | 188 | 3.5 | 37 | 40 | 18 |
| FGA-3 | 173 | 4.1 | 37 | 40 | 17 |
| FGA-4 | 229 | 3.3 | 41 | 60 | 20 |
| FGA-5 | 252 | 5.0 | 35 | 60 | 19 |
| FGA-6 | 251 | 4.0 | 36 | 60 | 20 |
| FGA-7 | 214 | 2.0 | 47 | 50 | 16 |
| FGA-8 | 258 | 4.8 | 37 | 70 | 16 |
| FGA-9 | 261 | 4.2 | 42 | 80 | 16 |
| FGA-10 | 271 | 3.9 | 40 | 90 | 15 |
| Average | 230 | 3.9 | 39 | 59 | 17 |
| Standard | 34 | 0.8 | 4.0 | 17 | 2.0 |
| Deviation | | | | | |

TABLE 5
Summary of mechanical properties of
16x GDVIEV proteins disclosed herein.

| | Peak | | Strain | | |
|---|---|---|---|---|---|
| | stress/ | Modulus/ | at break/ | Toughness/ | Diameter/ |
| | MPa | GPa | % | MJ*m⁻³ | μm |
| GDV-1 | 296 | 6.0 | 24 | 40 | 12 |
| GDV-2 | 258 | 3.2 | 35 | 60 | 12 |
| GDV-3 | 322 | 6.7 | 22 | 50 | 11 |
| GDV-4 | 311 | 7.3 | 23 | 50 | 11 |
| GDV-5 | 279 | 4.7 | 28 | 50 | 12 |
| GDV-6 | 228 | 5.3 | 45 | 70 | 10 |
| GDV-7 | 208 | 5.8 | 31 | 70 | 10 |
| GDV-8 | 198 | 4.9 | 30 | 70 | 10 |
| GDV-9 | 394 | 4.4 | 33 | 60 | 10 |
| GDV-10 | 309 | 5.1 | 38 | 70 | 10 |
| Average | 280 | 5.3 | 31 | 59 | 11 |
| Standard | 60 | 1.2 | 7.2 | 11 | 0.74 |
| Deviation | | | | | |

TABLE 6
Summary of mechanical properties of
16x polyA proteins disclosed herein.

| | Peak | | Strain | | |
|---|---|---|---|---|---|
| | stress/ | Modulus/ | at break/ | Toughness/ | Diameter/ |
| | MPa | GPa | % | MJ*m⁻³ | μm |
| polyA-1 | 74 | 1.9 | 40 | 25 | 19 |
| polyA-2 | 98 | 3.4 | 54 | 40 | 17 |
| polyA-3 | 77 | 2.4 | 43 | 26 | 17 |
| polyA-4 | 104 | 3.1 | 59 | 50 | 17 |
| polyA-5 | 73 | 2.6 | 84 | 50 | 16 |
| polyA-6 | 78 | 2.1 | 87 | 50 | 18 |
| Average | 84 | 2.6 | 61 | 40 | 17 |
| Standard | 14 | 0.6 | 20 | 12 | 1.1 |
| Deviation | | | | | |

While the recombinant spider silk fiber displayed an ultimate tensile strength of 82±11 MPa, close to previous results using spidroin with similar molecular weight, all three polymeric amyloid fibers had significantly higher initial modulus and ultimate tensile strength. The ultimate tensile strength for 16× GDV, 16× KLV, and 16× FGA reached 280±60 MPa, 243±16 MPa, and 230±34 MPa, respectively, presenting 2.4- 2.0-, and 1.8-fold enhancement from the 16× recombinant silk fiber. The enhanced fiber modulus and strength does not result in comprised toughness, which increased by 48%, 160%, and 48% for 16× GDV, 16× KLV, and 16× FGA fibers, respectively, compared to that of the 16× recombinant silk fiber.

To understand the origin of enhanced mechanical properties of amyloid fibers, synchrotron-based wide-angle X-ray diffraction (WAXD) was used to study the structures of polymeric amyloid fibers and the recombinant silk fibers. Two broad but distinct equatorial reflections were observed in the two-dimensional diffraction images of all fibers (see FIG. 6A-FIG. 6D), characteristic of semi-crystalline materials. The equatorial 1D profile of 16× recombinant spider silk was deconvoluted into two crystalline peaks, two broader peaks above 1 Å⁻¹ as seen in natural spider silks, and one amorphous peak below 1 Å⁻¹. This amorphous peak was observed in other regenerated silk fibers from wet-spinning but not in natural silk fibers, representing loosely packed β-sheet, potential defects from artificial spinning. The two crystalline peaks have d-spacings of 0.45 nm (120) and 0.57 nm (200), consistent with natural spider silks, and represent distance between adjacent β-strands and β-sheets, respectively.

Crystallite size of 16× recombinant silk was estimated to be 1.4 nm and 3.7 nm along the inter-sheet and inter-strand axes, respectively, slightly smaller than those of natural silk fiber. In contrast, the (200) crystalline peak of 16× amyloid fibers appeared at a different d spacing of 0.91 nm, indicating a larger inter-sheet distance. This larger inter-sheet distance is caused by the bulky sidechains in amyloid peptides and is consistent with diffraction patterns of self-assembled amyloid nanofibrils. Crystallite size along the inter-sheet axis was estimated to be 0.91 nm, indicating two layers of β-sheets packed together in every ordered crystallite, also consistent with previous crystal structures of amyloid peptides. Thus, the polymeric amyloid fibers described herein maintain some of the cross-β structure characteristic to amyloids. Crystallite size along the inter-strand axis was estimated to be 3.7 nm, similar to that of 16× recombinant silk fiber.

Next, the crystallinity of each fiber was estimated using the deconvoluted peaks (see Table 2 and Table 3). All polymeric amyloid fibers displayed a drastically higher crystallinity from 15-19%, compared to 4.2% in 16× recombinant silk fiber. Further analyzing the azimuthal 1D profiles of the two reflections allowed for the orientation parameter to be calculated. The crystallites are highly orientated in all fibers with β-strands aligned in parallel with fiber axis. The orientation parameter of crystallite ƒ_{crystal} ranges from 0.85 to 0.89, close to natural silk fibers. The amorphous components are weakly oriented with ƒ_{disorder} ranging from 0.14 to 0.4. The orientation parameters of the amorphous domains are significantly lower than that of natural silk fibers (ranging from 0.45 to 0.81), probably due to difference in spinning processes. Overall, these results suggest that recombinant silk fiber from wet-spinning has low crystallinity, which can be dramatically enhanced by replacing the poly alanine sequence with amyloid peptides, therefore providing higher strengths and moduli to protein fibers under similar MW.

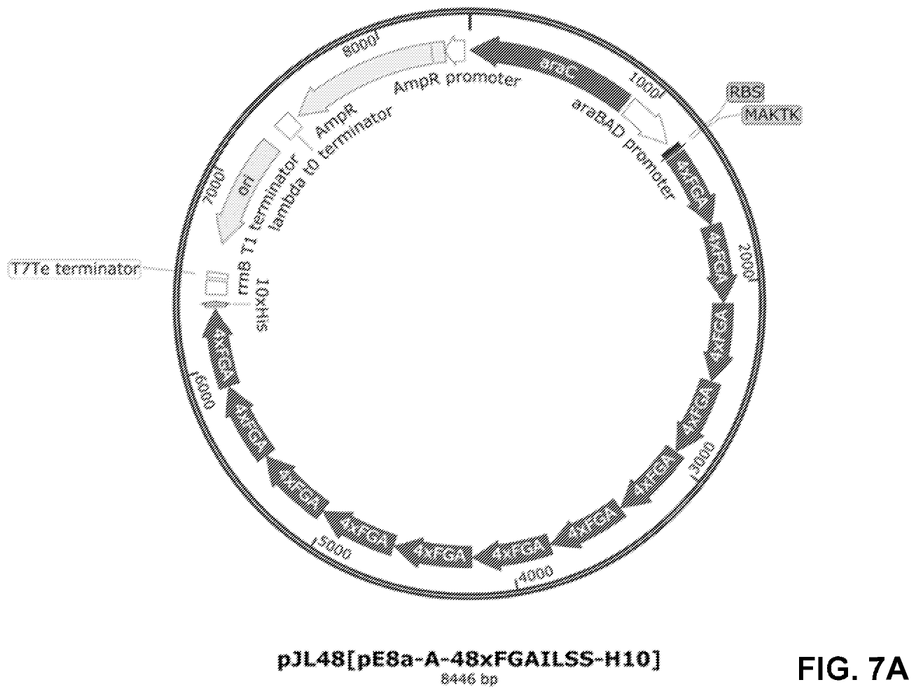

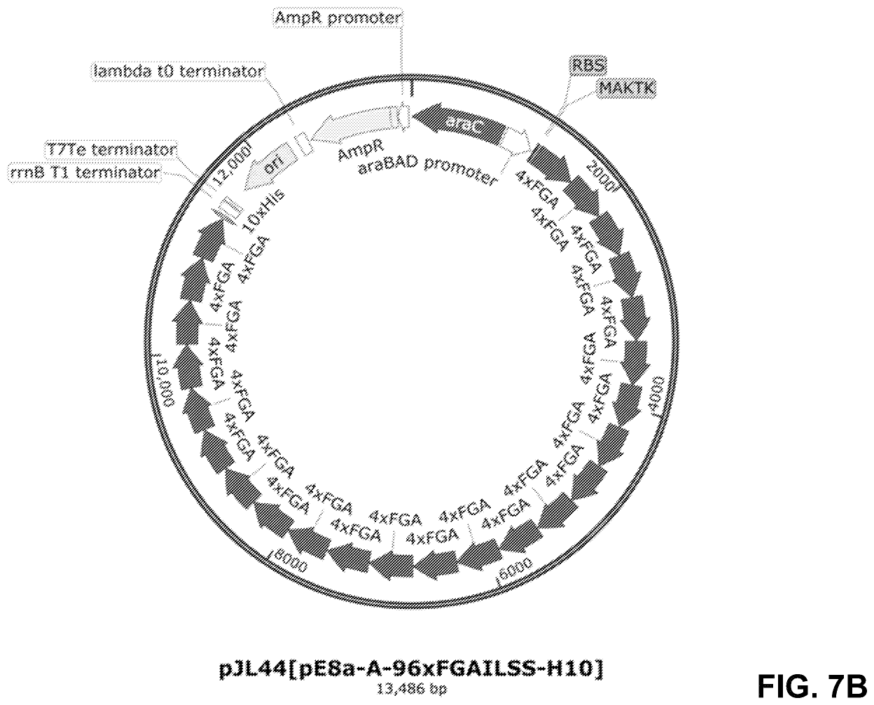

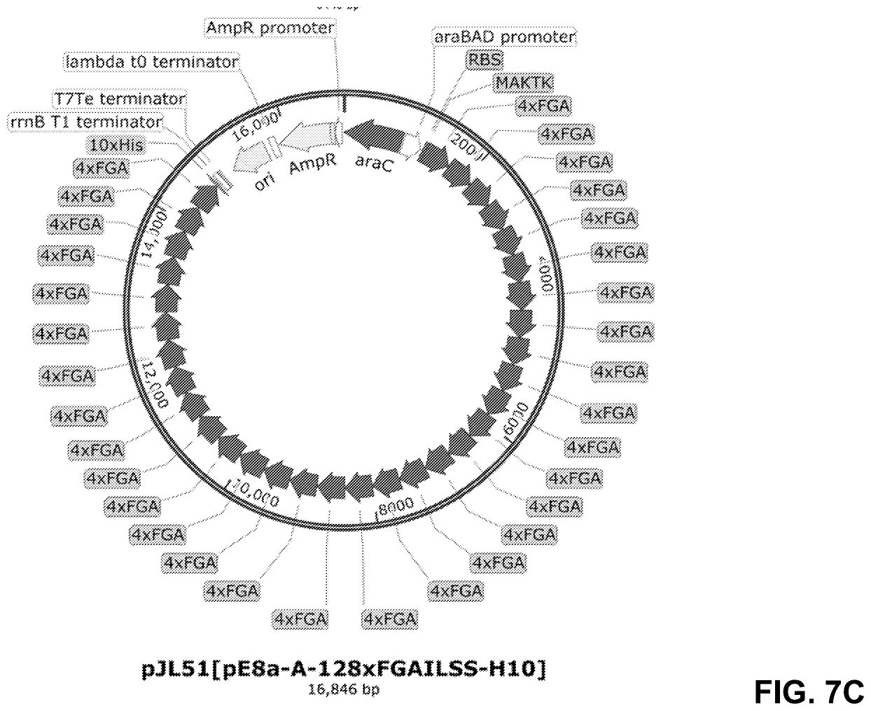

Previous works on silk fiber have demonstrated a positive correlation between the tensile strength of silk fiber with the MW of spidroin. With the enhanced crystallinity from amyloid sequence, high mechanical properties were next obtained by producing high MW polymeric amyloid proteins. Due to highly repetitive alanine sequences, recombinant silk protein with 128 repeats failed to express in engineered E. coli. An additional ligation step had to be used to obtain recombinant spidroins with higher than 96 repeats, which lowered the overall protein yield and incurred additional cost in protein production. Compared to poly-alanine sequences in spidroin, sequence repetitiveness of the amyloid peptides is drastically lower, thus easing production in a heterologous host (see FIG. 7A-FIG. 7C).

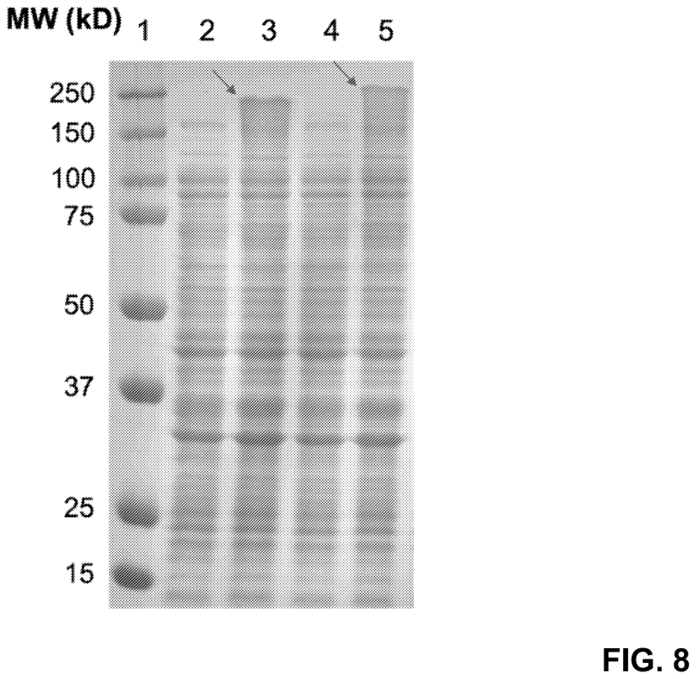

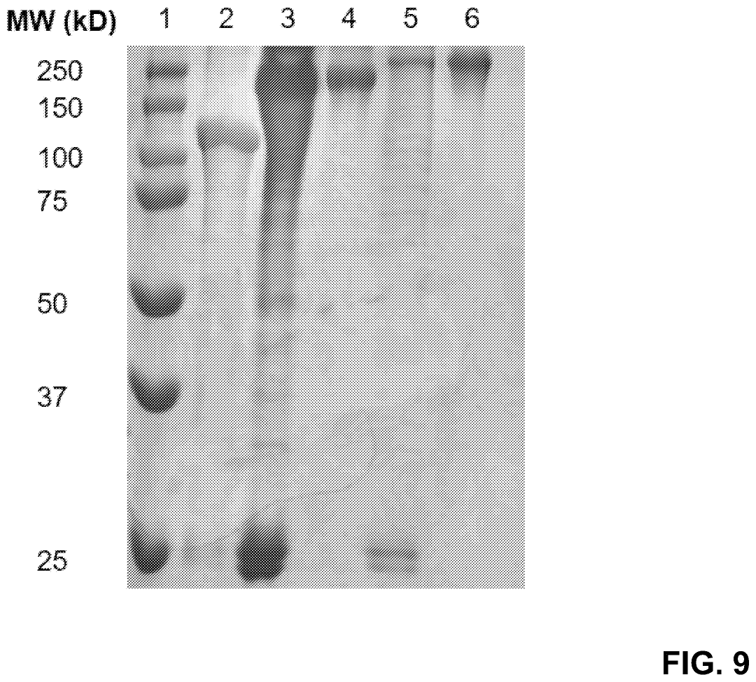

High MW polymeric FGAILSS proteins were constructed with 48× (143 kDa), 96× (284 kDa) and 128× (378 kDa) tandem repeats (see FIG. 8). All polymeric proteins were overexpressed in the engineered E. coli host. Surprisingly, these high MW proteins are able to be purified by affinity chromatography (see FIG. 9), instead of the laborious selective-precipitation required for high MW recombinant spidroin, thus further simplifying fabrication process and reducing production cost.

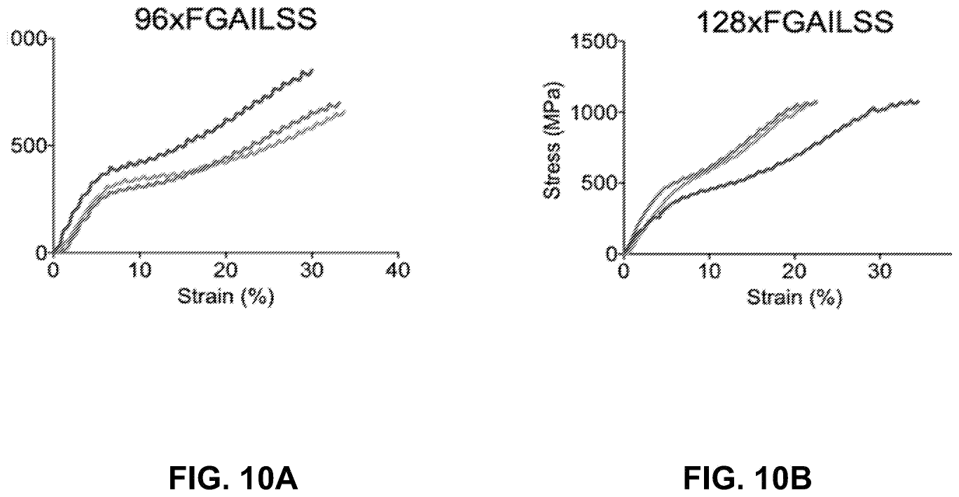

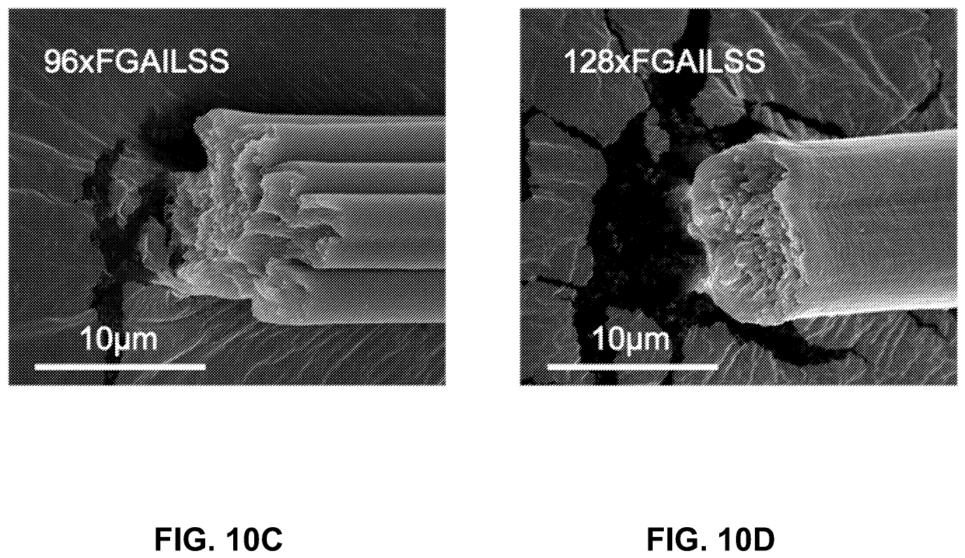

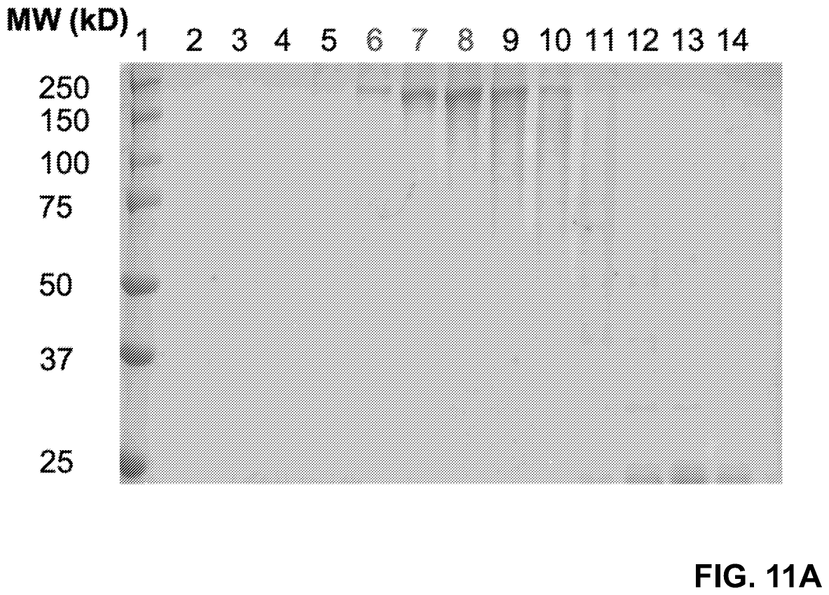

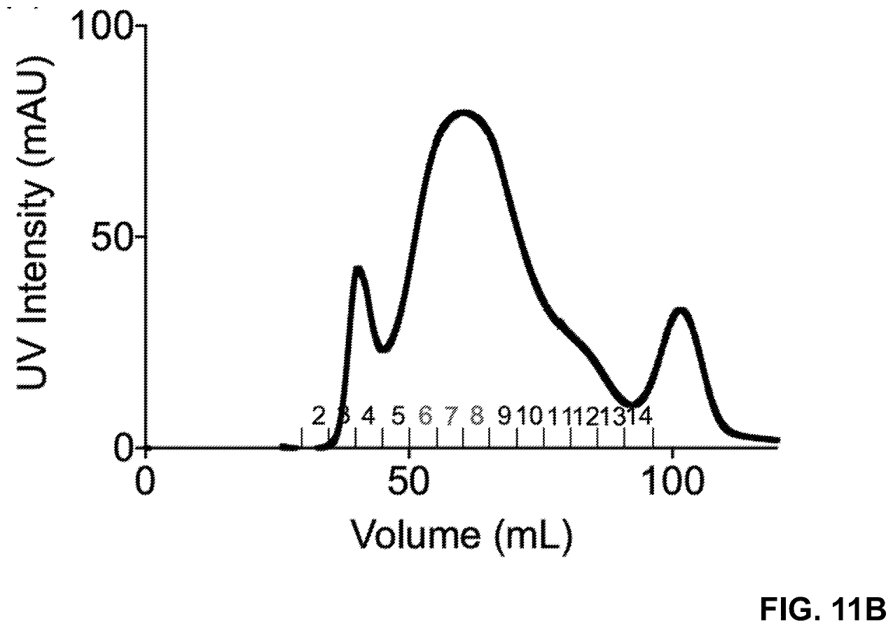

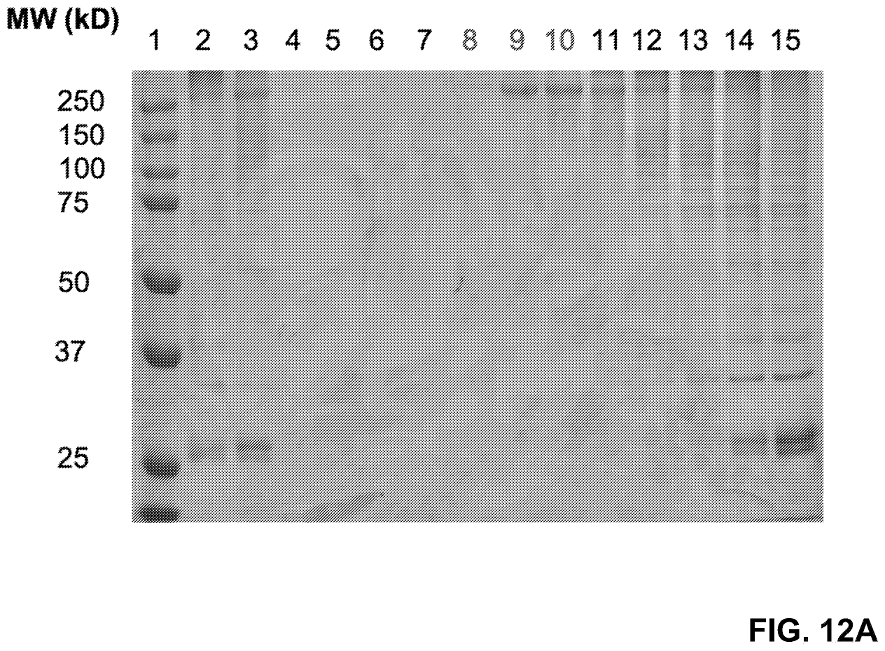

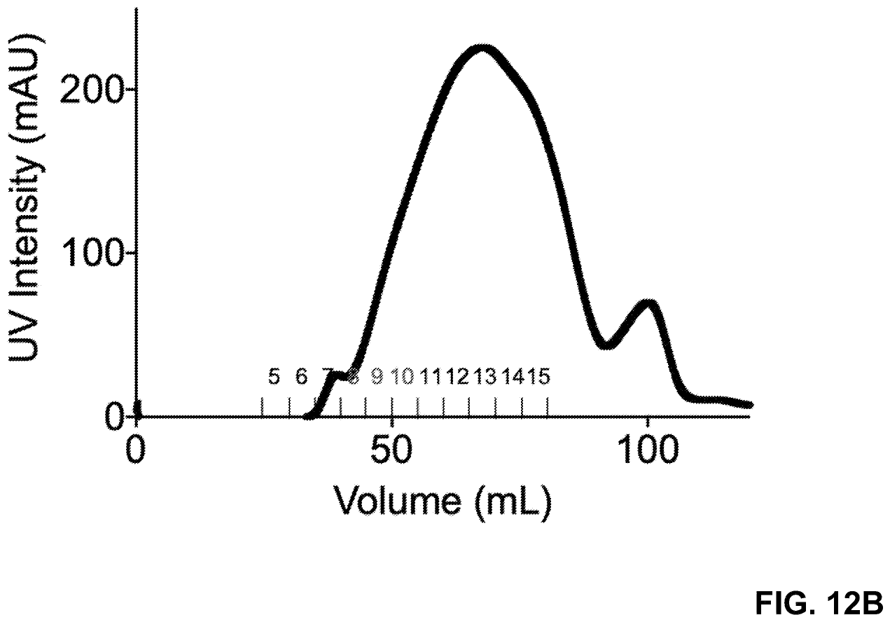

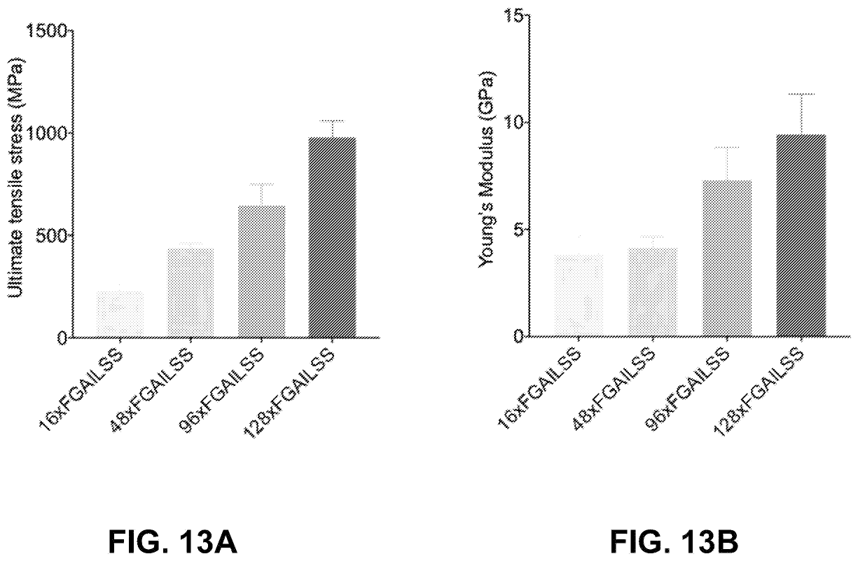

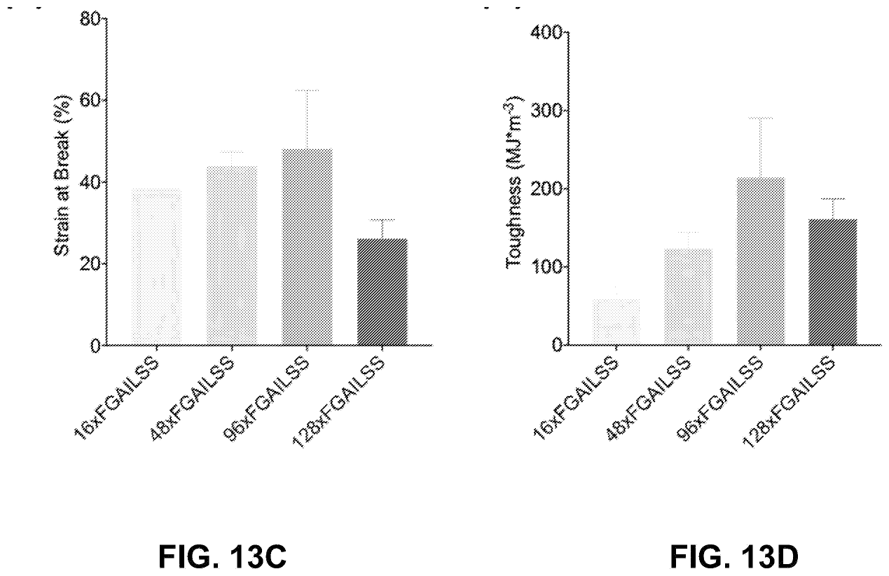

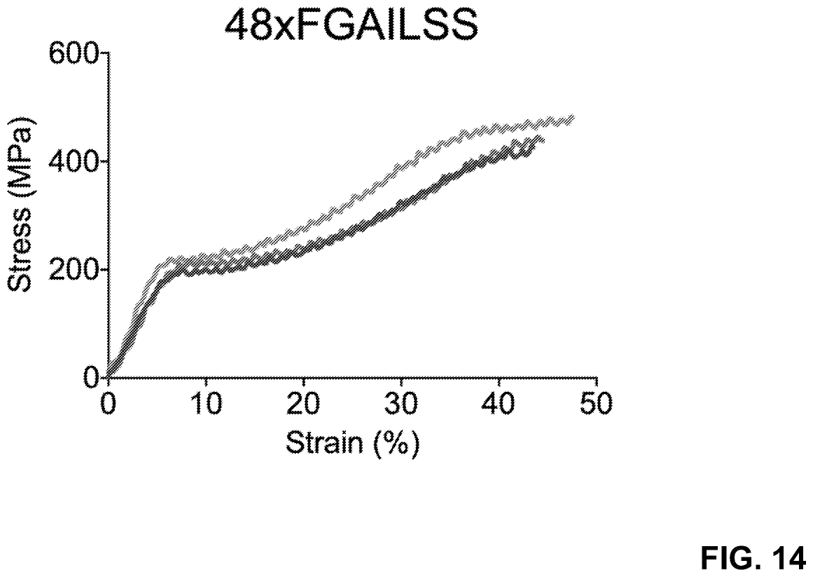

The purified high MW proteins were spun into fibers using the same protocol for 16× proteins (see FIG. 10A-FIG. 10D, FIG. 11A-FIG. 11B, FIG. 12A-FIG. 12B). Results from tensile testing of the high MW FGAILSS fibers confirmed the positive correlation between polymer MW and fiber strength (see FIG. 13A-FIG. 13D, FIG. 14). The ultimate tensile stress of 48×-, 96×-, and 128× FGAILSS fibers are 0.44±0.02 GPa, 0.65±0.11 GPa and 0.98±0.08 GPa, respectively (see Table 7-Table 10).

TABLE 7
Summary of mechanical properties of 48x
FGAILSS hybrid proteins disclosed herein.

| | Peak | | Strain | | |
|---|---|---|---|---|---|
| | stress/ | Modulus/ | at break/ | Toughness/ | Diameter/ |
| | MPa | GPa | % | MJ*m⁻³ | μm |
| 48FGA-1 | 432 | 3.7 | 41 | 120 | 14 |
| 48FGA-2 | 422 | 4.7 | 49 | 130 | 14 |
| 48FGA-3 | 428 | 3.7 | 43 | 110 | 14 |
| 48FGA-4 | 445 | 3.8 | 43 | 120 | 14 |
| 48FGA-5 | 482 | 4.9 | 47 | 160 | 14 |
| 48FGA-6 | 417 | 4.1 | 40 | 100 | 14 |
| Average | 438 | 4.2 | 44 | 123 | 14 |
| Standard | 24 | 0.5 | 3.4 | 21 | 0.27 |
| Deviation | | | | | |

TABLE 8
Summary of mechanical properties of 96x
FGAILSS proteins disclosed herein.

| | Peak | | Strain | | |
|---|---|---|---|---|---|
| | stress/ | Modulus/ | at break/ | Toughness/ | Diameter/ |
| | MPa | GPa | % | MJ*m⁻³ | μm |
| 96FGA-1 | 560 | 4.7 | 56 | 190 | 10 |
| 96FGA-2 | 689 | 7.0 | 33 | 140 | 10 |
| 96FGA-3 | 731 | 6.8 | 32 | 140 | 11 |
| 96FGA-4 | 533 | 6.2 | 48 | 200 | 11 |
| 96FGA-5 | 700 | 10.0 | 74 | 400 | 11 |
| 96FGA-6 | 558 | 7.2 | 61 | 260 | 13 |
| 96FGA-7 | 866 | 9.5 | 30 | 160 | 9.1 |
| 96FGA-8 | 571 | 7.1 | 55 | 230 | 13 |
| 96FGA-9 | 678 | 7.6 | 39 | 190 | 11 |
| 96FGA-10 | 577 | 7.1 | 53 | 230 | 13 |
| Average | 646 | 7.3 | 48 | 214 | 11 |
| Standard | 105 | 1.5 | 14 | 76 | 1.4 |
| Deviation | | | | | |

TABLE 9
Summary of mechanical properties of 128x
FGAILSS hybrid proteins disclosed herein.

| | Peak | | Strain | | |
|---|---|---|---|---|---|
| | stress/ | Modulus/ | at break/ | Toughness/ | Diameter/ |
| | MPa | GPa | % | MJ*m⁻³ | μm |
| 128FGA-1 | 1080 | 7.5 | 34 | 220 | 9.1 |
| 128FGA-2 | 971 | 9.4 | 28 | 160 | 9.6 |
| 128FGA-3 | 891 | 8.4 | 25 | 160 | 9.1 |
| 128FGA-4 | 901 | 9.3 | 22 | 140 | 8.8 |
| 128FGA-5 | 933 | 11.7 | 32 | 170 | 9.4 |
| 128FGA-6 | 916 | 7.4 | 24 | 150 | 8.8 |
| 128FGA-7 | 1074 | 12.7 | 22 | 150 | 8.8 |
| 128FGA-8 | 1067 | 9.0 | 22 | 140 | 8.3 |
| Average | 979 | 9.4 | 26 | 161 | 9.0 |
| Standard | 82 | 1.9 | 4.6 | 26 | 0.41 |
| Deviation | | | | | |

TABLE 10
Summary of mechanical properties of natural and proteinaceous
silks reported previously and disclosed herein.

| | Peak stress/ | Toughness/ | Diameter/ |
|---|---|---|---|
| | MPa | MJ*m⁻³ | μm |
| Natural N. clavipes | 965 ± 217 | 111 ± 30 | 4.7 ± 1.3 |
| dragline silk | | | |
| Natural A. trifasciata | 890 ± 130 | 100 ± 40 | ~3 |
| dragline silk | | | |
| Natural A. sericatus | 710 | 106 | N/A |
| dragline silk | | | |
| Natural A. trifasciata | 483 ± 34 | 150 ± 12 | N/A |
| minor ampullate silk | | | |
| Synthetic 96x | 508 ± 108 | N/A | N/A |
| MaSp1/MaSp2 4:1 mix | 47 ± 22 | 42 ± 8 | N/A |
| 11RPC | 308 ± 57 | N/A | N/A |
| NT2RepCT | 162 ± 8 | 45 ± 7 | 12 ± 2 |
| eADF3(AQ)12NR3 | 54 ± 16 | 2 ± 0.8 | 39 ± 6 |
| SRT-ELP Cys36x | 603 ± 18 | 113 ± 15 | N/A |
| rcSp2 | 330 ± 20 | 62 ± 7 | 36 ± 2 |
| Synthetic 192x | 1031 ± 111 | 114 ± 51 | 5.3 ± 1.7 |
| 128x FGAILSS | 979 ± 82 | 161 ± 26 | 9.0 ± 0.4 |

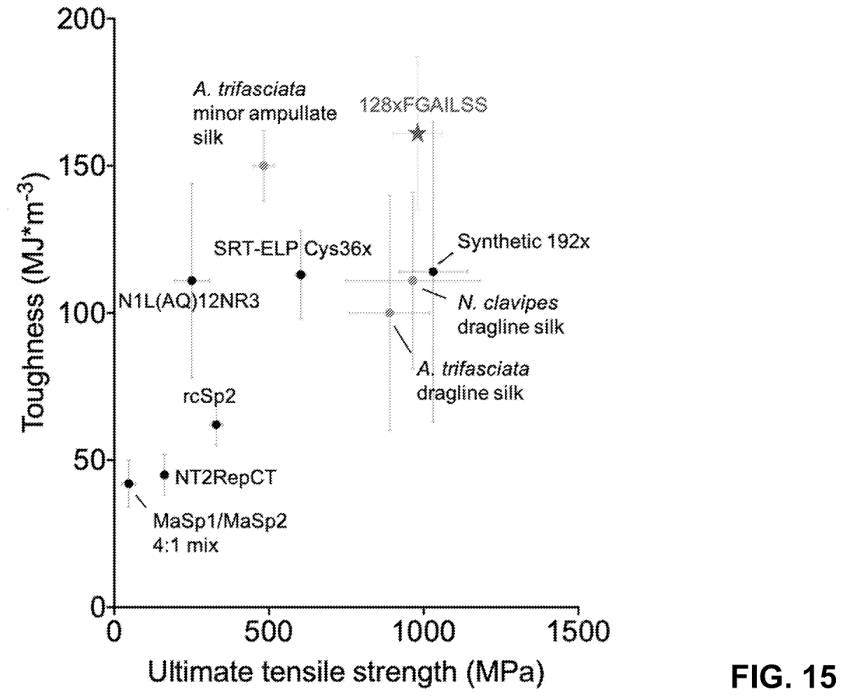

Strength and toughness of the 128× FGAILSS fiber described herein have surpassed most recombinant silk fibers and other protein fibers (see FIG. 15, Table 10). The tensile strength of the 128× FGAILSS fiber is even higher than reported dragline spider silk fibers of Abantiades sericatus and comparable to those of N. clavipes and Argiope trifasciata, while displaying higher toughness. Overall, the ease of bioproduction and purification as well as high mechanical properties make the 128× FGAILSS fiber a much more attractive candidate for biosynthetic high-strength fiber than recombinant silk proteins.

### Discussion

Taken together, the results demonstrate the feasibility of combining amyloid-peptides with spidroins in pursuit of strong recombinant silk materials. The results further demonstrate that the methods described herein are effective and applicable for amyloid sequences of all three amyloid structural categories. The engineered hybrid fibers displayed better mechanical properties than the original silk sequences, which not only addressed the potential of amyloids to serve directly as strong materials but also enables recombinant silk production. By introducing foreign sequences, silk proteins were found to become easier to express and better in performance. Structural analyses revealed a higher degree of crystallinity within amyloid fibers compared with the poly-alanine recombinant silk from proteins of similar size.

Due to differences in the natural silk spinning process and the artificial wet-spinning of recombinant silk, recombinant silks were generally less crystalized and more poorly oriented compared with natural silks, which can explain the superiority of the latter. In some embodiments, the higher crystallinity in amyloid silks is attributed to elevated chances of loosely-packed β-sheets forming nano-crystals, compared with poly-alanine silks, caused by either a relatively higher inter-sheet distance in amyloid nano-crystallite or long-range intramolecular interactions between β-sheets such as electrostatic forces.

As a result, the overall alignment of the fiber is also improved, thus contributing to higher mechanical properties. The introduction of amyloid peptides also eased the expression of high MW proteins, allowing larger proteins to be produced which, after spinning, exhibited mechanical strength comparable to natural spider silk. Overall, for the first time amyloid proteins were demonstrated to serve as a strong macroscale material, paving the way for novel designs of proteinaceous silk materials.

The major challenge in recombinant silk fiber production was believed to be the molecular weight (MW) of recombinant proteins being not as high as those of natural spider silk proteins (spidroins). Recently, a 556 kDa recombinant silk protein was synthesized using synthetic biology approaches, whose molecular weight is approximately 80% higher than that of natural dragline spidroin. However, mechanical properties of its fiber are only on par with, but not stronger than, natural dragline spider silk, suggesting additional factors limiting mechanical properties of recombinant silk fibers.

### Materials and Methods

Chemicals and reagents. All chemicals and reagents used herein were purchased from MilliporeSigma (Burlington, MA) unless otherwise noted. Gel extraction kits and plasmid purification kits were purchased from iNtRON Biotechnology (Republic of Korea). FastDigest restriction enzymes and T4 DNA ligase were purchased from Thermo Fisher Scientific and used for all digestions and ligations following manufacturer-suggested protocols. Ni-NTA columns and ion exchange columns were purchased from GE Healthcare (Chicago, IL).

Strains and Growth Conditions. E. coli NEB10β strain was used in all plasmid cloning and protein expression. For all cloning, E. coli cells were cultured in Luria broth media (LB) containing 10 g/L tryptone, 5 g/L yeast extract, 10 g/L NaCl, and appropriate antibiotics (50 μg/mL kanamycin, 100 μg/mL ampicillin or 30 μg/mL chloramphenicol) with pH adjusted to 7.5. Terrific broth media containing 20 g/L tryptone, 24 g/L yeast extract, 0.4% glycerol, and phosphate buffer (0.017 M KH₂PO₄ and 0.072 M K₂HPO₄) with appropriate antibiotics were used for protein expression.

Plasmid Construction. DNA sequences encoding 4× tandem repeats were codon optimized for E. coli production using previous approaches. Designed DNA sequences were chemically synthesized by Integrated DNA Technologies, Inc. (Coralville, IA). Each DNA sequence was incorporated into a standard expression vector with an arabinose-inducible PBAD promoter or an isopropyl β-d-1-thiogalactopyranoside (IPTG)-inducible P_{Lac} promoter. A recursive digestion and ligation process utilizing NheI/BcuI restriction sites was performed repeatedly to obtain higher tandem repeats. All plasmids were individually transformed to E. coli NEB10β competent cells for protein production. To facilitate overexpression of high molecular weight proteins (96× FGAILSS and 128× FGAILSS) with high glycine content, an extra plasmid encoding the glycyltRNA was co-transformed (FIG. 7B and FIG. 7C).

Protein Expression on Shake Flasks. A single colony of E. coli transformed with a polymeric amyloid plasmid was cultured in TB medium at 37° C. on an orbital shaker. The culture was then used to inoculate fresh TB medium, which was allowed to grow to OD600 of 3-5. The culture was then induced by addition of 0.04% arabinose or 1 mM IPTG and was continued to grow at 30° C. for 6 hours. Cells were then pelleted by centrifugation, and cell pellets were stored at −80° C. until use.

Protein purification. Cell pellets were lysed in buffer A (6 M guanidine hydrochloride, 300 mM NaCl, 50 mM K2HPO4, pH=8.0) for 12 h at 4° C. under constant stirring followed by centrifugation. The supernatant was loaded to a Ni-NTA column and was sequentially washed by buffer B (8 M urea, 300 mL NaCl, 50 mM K₂HPO₄, pH=8.0) with 0, 20 mM, and 50 mM of imidazole. Polymeric amyloid proteins were then eluted with buffer B containing 300 mM imidazole. For 96× and 128× proteins, an extra size exclusion chromatography (SEC) purification was used to remove low molecular weight impurities. SEC purifications were performed on an AKTA Pure Chromatography System (GE Healthcare Life Sciences) using a HiPrep 16/60 Sephacryl S-400 HR column. Proteins were separated with an isocratic elution using buffer B at a flow rate of 1 mL/min. All purified proteins were dialyzed against 1% acetic acid, lyophilized, and stored at −80° C. until use.

SDS-PAGE and Purity Analysis. All SDS-PAGE gels were 1 mm thick and discontinuous with 5% stacking gel on the top and indicated percentages separation gels on the bottom. Samples were prepared in Laemmli sample buffer (2% SDS, 10% glycerol, 60 mM Tris pH 6.8, 0.01% bromophenol blue, 100 μM DTT). Gels were run on Mini-PROTEAN Tetra Cells (Bio-Rad) in 1× Tris-glycine SDS buffer (25 mM Tris base, 250 mM glycine, 0.1% w/v SDS), until just before the dye front exited the gel.

Light Microscopy. Fiber diameters were measured using a Zeiss Axio Observer ZI inverted microscope equipped with a phase contrast 20× objective lens and quantified using the Axiovision LE software (Zeiss).

Scanning Electron Microscopy (SEM). Fibers after tensile tests are mounted onto a sample holder using conductive tapes. The sample holder was sputter coated with 10 nm gold using a Leica EM ACE600 high-vacuum sputter coater (Leica Microsystems). Fibers were imaged with Nova NanoSEM 230 field emission scanning electron microscope (Field Electron and Ion Company, FEI) at an accelerating voltage of 10 kV.

Fiber Spinning and Tensile Testing. The spinning protocol for fiber spinning was adapted from previous methods for spinning recombinant spider silk with some modifications. Lyophilized protein powders were dissolved in HFIP to prepare spinning dopes to a concentration of 12.5% w/v. Dopes were then loaded into a Hamilton syringe (Hamilton Robotics) and slowly extruded into a 95% v/v methanol bath by a Harvard Apparatus Pump 11 Elite syringe pump (Harvard Apparatus) at a rate of 10 μL/min. Extruded fibers were transferred to a 75% v/v methanol bath and gently extended right before fracture for 4-6 times of their original lengths. Fibers post-extension were removed from the methanol bath and ventilated until dry. Tensile tests were conducted on an MTS Criterion Model 41 universal test frame fitted with a 1 N load cell (MTS Systems Corporation) at a relative humidity of 20% and temperature of 25° C. (room temperature), with a constant pulling speed of 10 mm/min. Stress-strain curves were recorded by the MTS TW Elite test suite at a sampling rate of 50 Hz. Mechanical properties of fibers were calculated by the MTS system based on the stress-strain curve it recorded and the diameter measured under optical microscope.

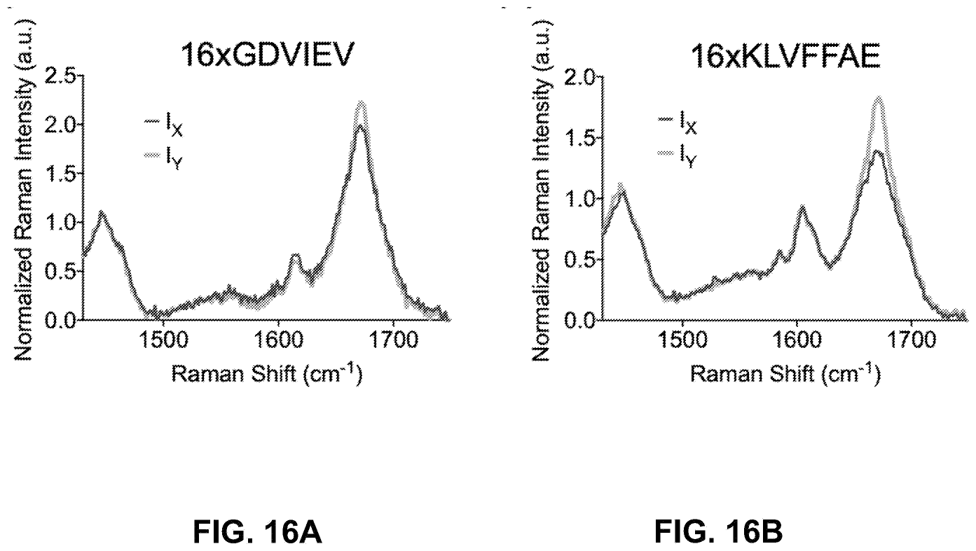

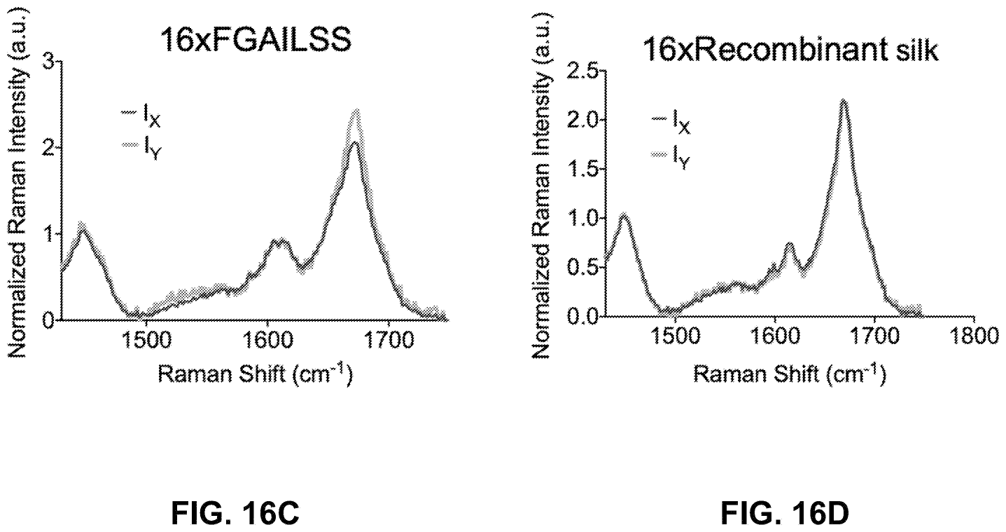

Polarized Raman Spectroscopy Analysis. A fiber sample was fixed on a glass slide by tape. Raman spectra were acquired with a Renishaw RM1000 InVia confocal Raman spectrometer (Renishaw) coupled to a Leica DM LM microscope with rotating stage (Leica Microsystems). Fibers were irradiated by a 514 nm argon laser with polarization fixed along the x-axis and focused through a 50× objective (NA=0.75). Spectra were recorded from 1150 to 1750 cm^{31 1} with an 1800 lines/mm grating, both perpendicular (IX) and parallel (IY) to the fiber axis (see FIG. 16A-FIG. 16D). For each acquisition, a total of 16 spectra were accumulated, each for 10 s. All fibers remained intact after acquisition with no visual sign of degradation under the incident laser. Spectra collected were analyzed with the Fityk 0.9.8 software. Baseline subtraction is accomplished using a built-in Fityk automatic convex hull algorithm. All spectra were normalized to the intensity of the 1450 cm⁻¹ peak, which arises from CH₃ asymmetric stretching and CH₂ bending.

Statistical Analysis. Statistical analyses, including student t-test and ANOVA, were conducted with Prism 8 (GraphPad Software).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that vary depending upon the desired properties sought to be obtained by a particular embodiment. In some embodiments, the numerical parameters are be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) are construed to cover both the singular and the plural, unless specifically noted otherwise. In some embodiments, the term “or” as used herein, including the claims, is used to mean “and/or” unless explicitly indicated to refer to alternatives only or to refer to the alternatives that are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and may also cover other unlisted steps. Similarly, any composition or device that “comprises,” “has” or “includes” one or more features is not limited to possessing only those one or more features and may cover other unlisted features.

All methods described herein are performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member is referred to and claimed individually or in any combination with other members of the group or other elements found herein. One or more members of a group are included in, or deleted from, a group for reasons of convenience or patentability. When any such inclusion or deletion occurs, the specification is herein deemed to contain the group as modified thus fulfilling the written description of all Markush groups used in the appended claims.

To facilitate the understanding of the embodiments described herein, a number of terms are defined below. The terms defined herein have meanings as commonly understood by a person of ordinary skill in the areas relevant to the present disclosure. Terms such as “a,” “an,” and “the” are not intended to refer to only a singular entity, but rather include the general class of which a specific example may be used for illustration. The terminology herein is used to describe specific embodiments of the disclosure, but their usage does not delimit the disclosure, except as outlined in the claims.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system for synthesizing a recombinant polymeric amyloid in vivo, the system comprising:
a host cell; and
a plasmid encoding tandem repeats of an amyloid peptide and a glycine-rich linker peptide.

2. The system of claim 1, wherein the glycine-rich linker peptide is a silk amino acid sequence or other flexible peptide sequence.

3. The system of claim 1, wherein the amyloid peptide is selected from a full amyloid peptide and an amyloid peptide fragment.

4. The system of claim 1, wherein the amyloid peptide is selected from a parallel amyloid, an antiparallel homo-facial amyloid, and an antiparallel hetero-facial amyloid.

5. The system of claim 1, wherein the host cell is a microbial cell.

6. The system of claim 1, wherein the plasmid encodes at least about 16 tandem repeats.

7. The system of claim 1, wherein the amyloid peptide is encoded by an amino acid sequence having at least about 50% similarity with a β-sheet-forming amyloid peptide.

8. A method of synthesizing a recombinant polymeric amyloid, the method comprising:
synthesizing tandem repeats of an amyloid peptide and a glycine-rich linker peptide in vivo in a heterologous host.

9. The method of claim 8, wherein the amyloid peptide is selected from a parallel amyloid, an antiparallel homo-facial amyloid, and an antiparallel hetero-facial amyloid.

10. The method of claim 8, wherein the glycine-rich linker peptide is a silk amino acid sequence or other flexible peptide sequence.

11. The method of claim 8, further comprising purifying the recombinant polymeric amyloid.

12. The method of claim 8, further comprising spinning the recombinant polymeric amyloid into fibers.

13. The method of claim 8, wherein the recombinant polymeric amyloid has a molecular weight of at least about 45 kDa.

14. A recombinant polymeric amyloid fiber comprising:
a plurality of polymeric amyloid fibrils, each comprising a plurality of β-sheet crystals, wherein the β-sheet crystals comprise tandem repeats of an amyloid peptide and a glycine-rich linker peptide, and wherein
the plurality of β-sheet crystals are aligned in parallel with a fiber axis.

15. The recombinant polymeric amyloid fiber of claim 14, wherein the amyloid peptide is selected from a parallel amyloid, an antiparallel homo-facial amyloid, and an antiparallel hetero-facial amyloid.

16. The recombinant polymeric amyloid fiber of claim 14, wherein the amyloid peptide is encoded by an amino acid sequence having at least about 50% similarity with a β-sheet-forming amyloid peptide.

17. The recombinant polymeric amyloid fiber of claim 14, wherein the glycine-rich linker peptide is a silk amino acid sequence or other flexible peptide sequence.

18. The recombinant polymeric amyloid fiber of claim 14, wherein the fiber has a crystallinity of at least about 10%.

19. The recombinant polymeric amyloid fiber of claim 14, wherein the β-sheet crystals comprise at least about 16 tandem repeats.

20. The recombinant polymeric amyloid fiber of claim 14, wherein the β-sheet crystals comprise at least about 90 tandem repeats.

21. (canceled)


SEQUENCE LISTING (10 sequences)

SEQ ID NO: 1 - protein, 6 aa, Homo sapiens
GDVIEV

SEQ ID NO: 2 - protein, 7 aa, Artificial Sequence
  Synthetic amyloid peptide
KLVFFAE

SEQ ID NO: 3 - protein, 7 aa, Homo sapiens
FGAILSS

SEQ ID NO: 4 - protein, 565 aa, Artificial Sequence
  Synthetic 16xGDVIEV
MAKTKGTASGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSGRGGLGGQGAGGDVIEVGGAGQGGYGGLGSQGTSSGHHHHHHHHHH

SEQ ID NO: 5 - protein, 581 aa, Artificial Sequence
  Synthetic 16xKLVFFAE
MAKTKGTASGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSGRGGLGGQGAGKLVFFAEGGAGQGGYGGLGSQGTSSGHHHHHHHHHH

SEQ ID NO: 6 - protein, 581 aa, Artificial Sequence
  Synthetic 16xFGAILSS
MAKTKGTASGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSSGHHHHHHHHHH

SEQ ID NO: 7 - protein, 549 aa, Artificial Sequence
  Synthetic 16xRecombinant silk
MAKTKGTASGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSGRGGLGGQGAGAAAAAGGAGQGGYGGLGSQGTSSGHHHHHHHHHH

SEQ ID NO: 8 - protein, 1701 aa, Artificial Sequence
  Synthetic 48xFGAILSS
MAKTKGTASGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSSGHHHHHHHHHH

SEQ ID NO: 9 - protein, 3381 aa, Artificial Sequence
  Synthetic 96xFGAILSS
MAKTKGTASGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSSGHHHHHHHHHH

SEQ ID NO: 10 - protein, 4501 aa, Artificial Sequence
  Synthetic 128xFGAILSS
MAKTKGTASGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSGRGGLGGQGAGFGAILSSGGAGQGGYGGLGSQGTSSGHHHHHHHHHH